SEQUENCE LISTING (84 sequences)

SEQ ID NO: 1 - protein, 562 aa, Influenza A virus
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTEKGSNYPVAKGSYNNTSGEQMLIIWGVHHPNDEKEQRTLYQNVGTYVSVGTSTLNKRSTPDIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMIDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 2 - protein, 561 aa, Influenza A virus
MYKVVVIIALLGAVKGLDRICLGHHAVANGTIVKTLTNEQEEVTNATETVESTNLNKLCMKGRSYKDLGNCHPVGMLIGTPVCDPHLTGTWDTLIERENAIAHCYPGATINEEALRQKIMESGGISKMSTGFTYGSSITSAGTTKACMRNGGDSFYAELKWLVSKTKGQNFPQTTNTYRNTDTAEHLIIWGIHHPSSTQEKNDLYGTQSLSISVESSTYQNNFVPVVGARPQVNGQSGRIDFHWTLVQPGDNITFSDNGGLIAPSRVSKLTGRDLGIQSEALIDNSCESKCFWRGGSINTKLPFQNLSPRTVGQCPKYVNQRSLLLATGMRNVPEVVQGRGLFGAIAGFIENGWEGMVDGWYGFRHQNAQGTGQAADYKSTQAAIDQITGKLNRLIEKTNTEFESIESEFSETEHQIGNVINWTKDSITDIWTYNAELLVAMENQHTIDMADSEMLNLYERVRKQLRQNAEEDGKGCFEIYHTCDDSCMESIRNNTYDHSQYREEALLNRLNINPVKLSSGYKDIILWFSFGESCFVLLAVVMGLVFFCLKNGNMRCTICI

SEQ ID NO: 3 - protein, 570 aa, Influenza A virus
MNTQIIVILVLGLSMVKSDKICLGHHAVANGTKVNTLTERGVEVVNATETVEITGIDKVCTKGKKAVDLGSCGILGTIIGPPQCDLHLEFKADLIIERRNSSDICYPGRFTNEEALRQIIRESGGIDKESMGFRYSGIRTDGATSACKRTVSSFYSEMKWLSSSMNNQVFPQLNQTYRNTRKEPALIVWGVHHSSSLDEQNKLYGTGNKLITVGSSKYQQSFSPSPGARPKVNGQAGRIDFHWMLLDPGDTVTFTFNGAFIAPDRATFLRSNAPSGIEYNGKSLGIQSDAQIDESCEGECFYSGGTINSPLPFQNIDSRAVGKCPRYVKQSSLPLALGMKNVPEKIRTRGLFGAIAGFIENGWEGLIDGWYGFRHQNAQGQGTAADYKSTQAAIDQITGKLNRLIEKTNKQFELIDNEFTEVEQQIGNVINWTRDSLTEIWSYNAELLVAMENQHTIDLADSEMNKLYERVRRQLRENAEEDGTGCFEIFHRCDDQCMESIRNNTYNHTEYRQEALQNRIMINPVKLSSGYKDVILWFSFGASCVMLLAIAMGLIFMCVKNGNLRCTICI

SEQ ID NO: 4 - protein, 560 aa, Influenza A virus
MNTQILILALVAIIPTNADKICLGHHAVSNGAKVNTLTERGVEVVNATETVERTNVPRICSKGKRTVDLGQCGLLGTITGPPQCDQFLEFSADLIIERREGNDVCYPGKFVNEEALRQILRKSGGIDKETMGFTYSGIRTNGATSACRRSGSSFYAEMKWLLSNTDNAAFPQMTKSYKNIRKDPALIIWGIHHSGSTAEQTKLYGSGNKLITVGSSNYQQSFVPSPGARPQVNGQSGRIDFHWLMLNPNDTVTFSFNGAFIAPDRASFLRGKSMGIQSEVQVDANCEGDCYHSGGTILSSLPFQNINSRTVGECPRYVKQESLLLATGMKNVPEIPKGRGLFGAIAGFIENGWEGLVDGWYGFRHQNAQGEGTAADYKSTQSAIDQITGKLNRLIEKTNQQFELIDNEFTEVEKQIGNVINWTRDSLTEMWSYNAELLVAMENQHTIDLADSEMNKLYERVRRQLRENAEEDGTGCFEIFHKCDDDCMASIRNNTYDHSKYREEAIQNRIQIDPVKLSSGYKDVILWFSFGASCFILLAIAMGLVFICVKNGNMRCTICI

SEQ ID NO: 5 - protein, 566 aa, Influenza A virus
MEARLLVLLCAFAATNADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADQKSTQNAIDGITNKVNSVIEKMNTQFTAVGKEFNNLERRIENLNKKVDDGFLDIWTYNAELLVLLENERTLDFHDSNVRNLYEKVKSQLKNNAKEIGNGCFEFYHKCDDACMESVRNGTYDYPKYSEESKLNREEIDGVKLESMGVYQILAIYSTVASSLVLLVSLGAISFWMCSNGSLQCRICI

SEQ ID NO: 6 - protein, 552 aa, Influenza A virus
DQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSSYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADQESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 7 - protein, 564 aa, Influenza A virus
MLSIVILFLLIAENSSQNYTGNPVICMGHHAVANGTMVKTLADDQVEVVTAQELVESQNLPELCPSPLRLVDGQTCDIINGALGSPGCDHLNGAEWDVFIERPNAVDTCYPFDVPEYQSLRSILANNGKFEFIAEEFQWNTVKQNGKSGACKRANVDDFFNRLNWLVKSDGNAYPFQNLTKINNGDYARLYIWGVHHPSTSTEQINLYKNNPGRVTVSTKTSQTSVVPDIGSRPLVRGQSGRVSFYWTIVEPGDLIVFNTIGNLIAPRGHYKLNNQKKSTILNTAIPIGSCVSKCHTDKGSLSTTKPFQNISRIAVGDCPRYVKQGSLKLATGMRNIPEKASRGLFGAIAGFIENGWQGLIDGWYGFRHQNAEGTGTAADLKSTQAAINQINGKLNRLIEKTNDKYHQIEKEFEQVEGRIQDLENYVEDTKIDLWSYNAELLVALENQHTIDVTDSEMNKLFERVRRQLRENAEDKGNGCFEIFHKCDNNCIESIRNGTYDHDIYRDEAIDNRFQIQGVKLTQGYKDIILWISFSISCFLLVALLLAFILWACQNGNIRCQICI

SEQ ID NO: 8 - protein, 566 aa, Influenza A virus
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 9 - protein, 566 aa, Influenza A virus
MIAIIILAIVASTSKSDKICIGYHANNSTTQVDTILEKNVTVTHSVELLESQKEERFCRVLNKAPLDLKGCTIEGWILGNPQCDILLGDQRWSYIVERPGAQNGICYPGILNELEELKALIGSGERVQRFEMFPKSTWAGVDTSRGVTKACPYISGSSFYGNLLWIIKTESAAYPVIKGTYNNTGSQPILYFWGVHHPPDTNEQNTLYGSGDRYVRMGTESMNFAKSPEIAARPAVNGQRGRIDYYWSVLKPGETLNVESNGNLIAPWYAYKFTSSNNKGAVFKSNLPIENCDAVCQTVAGALRTNKTFQNVSPLWIGECPKYVKSDSLRLATGLRNVPQAETRGLFGAIAGFIEGGWTGMIDGWYGYHHENSQGSGYAADKESTQKAIDGITNKVNSIIDKMNTQFEAVDHEFSNLERRVDNLNKRMEDGFLDVWTYNAELLVLLENERTLDLHDANVKNLYEKVKSQLRDNAKDLGNGCFEFWHKCDDECINSVKNGTYDYPKYQDESKLNRQEIDSVKLENLGVYQILAIYSTVSSGLVLVGLIIAMGLWMCSNGSMPCKICI

SEQ ID NO: 10 - protein, 565 aa, Influenza A virus
MAVKVLHLLIIVLGRYSIADKICIGYLSNNSSDTVDTLTENGVPVTSSIDLVETNHTGTYCSLNGISPIHLGDCSFEGWIVGNPSCATNINIREWSYLIEDPNAPNKLCFPGELDNNGELRHLFSGVNSFSRTELISPSKWGDVLDGVTASCLDKGASSFYRNLVWLVKQNDRYPVVRGDYNNTTGRDVLVLWGIHHPDTETTATKLYVNKNPYTLVSTKEWSKRYELEIGTRIGDGQRSWMKIYWHLMHPGERIMFESNGGLLAPRYGYIIEKYGTGRIFQSGIRMAKCNTKCQTSMGGVNTNKTFQNIERNALGDCPKYIKSGQLKLATGLRNVPSIGERGLFGAIAGFIEGGWPGLINGWYGFQHQNEQGTGIAADKASTQKAINEITTKINNIIEKMNGNYDSIRGEFNQVEKRINMLADRVDDAVTDIWSYNAKLLVLIENDRTLDLHDANVKNLHEQVKRALKNNAIDEGDGCFNLLHKCNDSCMETIRNGTYNHEDYREESQLKRQEIEGIKLKTEDNVYKVLSIYSCIASSIVMVGLILAFIMWACSSGNCRFNVCI

SEQ ID NO: 11 - protein, 565 aa, Influenza A virus
MEKFIAIATLASTNAYDRICIGYQSNNSTDTVNTLIEQNVPVTQTMELVETEKHPAYCNTDLGAPLELRDCKIEAVIYGNPKCDIHLKDQGWSYIVERPSAPEGMCYPGSVENLEELRFVFSSAASYKRIRLFDYSRWNVTRSGTSKACNASTGGQSFYRSINWLTKKEPDTYDFNEGAYVNNEDGDIIFLWGIHHPPDTKEQTTLYKNANTLSSVTTNTINRSFQPNIGPRPLVRGQQGRMDYYWGILKRGETLKIRTNGNLIAPEFGYLLKGESYGRIIQNEDIPIGNCNTKCQTYAGAINSSKPFQNASRHYMGECPKYVKKASLRLAVGLRNTPSVEPRGLFGAIAGFIEGGWSGMIDGWYGFHHSNSEGTGMAADQKSTQEAIDKITNKVNNIVDKMNREFEVVNHEFSEVEKRINMINDKIDDQIEDLWAYNAELLVLLENQKTLDEHDSNVKNLFDEVKRRLSANAIDAGNGCFDILHKCDNECMETIKNGTYDHKEYEEEAKLERSKINGVKLEENTTYKILSIYSTVAASLCLAILIAGGLILGMQNGSCRCMFCI

SEQ ID NO: 12 - protein, 565 aa, Influenza A virus
MEKTLLFAAIFLCVKADEICIGYLSNNSTDKVDTIIENNVTVTSSVELVETEHTGSFCSINGKQPISLGDCSFAGWILGNPMCDELIGKTSWSYIVEKPNPTNGICYPGTLESEEELRLKFSGVLEFNKFEVFTSNGWGAVNSGVGVTAACKFGGSNSFFRNMVWLIHQSGTYPVIKRTFNNTKGRDVLIVWGIHHPATLTEHQDLYKKDSSYVAVGSETYNRRFTPEINTRPRVNGQAGRMTFYWKIVKPGESITFESNGAFLAPRYAFEIVSVGNGKLFRSELNIESCSTKCQTEIGGINTNKSFHNVHRNTIGDCPKYVNVKSLKLATGPRNVPAIASRGLFGAIAGFIEGGWPGLINGWYGFQHRDEEGTGIAADKESTQKAIDQITSKVNNIVDRMNTNFESVQHEFSEIEERINQLSKHVDDSVVDIWSYNAQLLVLLENEKTLDLHDSNVRNLHEKVRRMLKDNAKDEGNGCFTFYHKCDNKCIERVRNGTYDHKEFEEESKINRQEIEGVKLDSSGNVYKILSIYSCIASSLVLAALIMGFMFWACSNGSCRCTICI

SEQ ID NO: 13 - protein, 564 aa, Influenza A virus
MEKFIILSTVLAASFAYDKICIGYQTNNSTETVNTLSEQNVPVTQVEELVHRGIDPILCGTELGSPLVLDDCSLEGLILGNPKCDLYLNGREWSYIVERPKEMEGVCYPGSIENQEELRSLFSSIKKYERVKMFDFTKWNVTYTGTSKACNNTSNQGSFYRSMRWLTLKSGQFPVQTDEYKNTRDSDIVFTWAIHHPPTSDEQVKLYKNPDTLSSVTTVEINRSFKPNIGPRPLVRGQQGRMDYYWAVLKPGQTVKIQTNGNLIAPEYGHLITGKSHGRILKNNLPMGQCVTECQLNEGVMNTSKPFQNTSKHYIGKCPKYIPSGSLKLAIGLRNVPQVQDRGLFGAIAGFIEGGWPGLVAGWYGFQHQNAEGTGIAADRDSTQRAIDNMQNKLNNVIDKMNKQFEVVNHEFSEVESRINMINSKIDDQITDIWAYNAELLVLLENQKTLDEHDANVRNLHDRVRRVLRENAIDTGDGCFEILHKCDNNCMDTIRNGTYNHKEYEEESKIERQKVNGVKLEENSTYKILSIYSSVASSLVLLLMIIGGFIFGCQNGNVRCTFCI

SEQ ID NO: 14 - protein, 566 aa, Influenza A virus
MALNVIATLTLISVCVHADRICVGYLSTNSSERVDTLLENGVPVTSSIDLIETNHTGTYCSLNGVSPVHLGDCSFEGWIVGNPACTSNFGIREWSYLIEDPAAPHGLCYPGELNNNGELRHLFSGIRSFSRTELIPPTSWGEVLDGTTSACRDNTGTNSFYRNLVWFIKKNTRYPVISKTYNNTTGRDVLVLWGIHHPVSVDETKTLYVNSDPYTLVSTKSWSEKYKLETGVRPGYNGQRSWMKIYWSLIHPGEMITFESNGGFLAPRYGYIIEEYGKGRIFQSRIRMSRCNTKCQTSVGGINTNRTFQNIDKNALGDCPKYIKSGQLKLATGLRNVPAISNRGLFGAIAGFIEGGWPGLINGWYGFQHQNEQGTGIAADKESTQKAIDQITTKINNIIDKMNGNYDSIRGEFNQVEKRINMLADRIDDAVTDIWSYNAKLLVLLENDKTLDMHDANVKNLHEQVRRELKDNAIDEGNGCFELLHKCNDSCMETIRNGTYDHTEYAEESKLKRQEIDGIKLKSEDNVYKALSIYSCIASSVVLVGLILSFIMWACSSGNCRFNVCI

SEQ ID NO: 15 - protein, 560 aa, Influenza A virus
METISLITILLVVTASNADKICIGHQSTNSTETVDTLTETNVPVTHAKELLHTEHNGMLCATSLGHPLILDTCTIEGLVYGNPSCDLLLGGREWSYIVERSSAVNGTCYPGNVENLEELRTLFSSASSYQRIQIFPDTTWNVTYTGTSRACSGSFYRSMRWLTQKSGFYPVQDAQYTNNRGKSILFVWGIHHPPTYTEQTNLYIRNDTTTSVTTEDLNRTFKPVIGPRPLVNGLQGRIDYYWSVLKPGQTLRVRSNGNLIAPWYGHVLSGGSHGRILKTDLKGGNCVVQCQTEKGGLNSTLPFHNISKYAFGTCPKYVRVNSLKLAVGLRNVPARSSRGLFGAIAGFIEGGWPGLVAGWYGFQHSNDQGVGMAADRDSTQKAIDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYREESRLERQKIEGVKLESEGTYKILTIYSTVASSLVLAMGFAAFLFWAMSNGSCRCNICI

SEQ ID NO: 16 - protein, 533 aa, Influenza A virus
MEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQNGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 17 - protein, 533 aa, Influenza A virus
MEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQNGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 18 - protein, 533 aa, Influenza A virus
MEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 19 - protein, 533 aa, Influenza A virus; identical to SEQ ID NO: 18

SEQ ID NO: 20 - protein, 565 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCR

SEQ ID NO: 21 - protein, 122 aa, Homo Sapiens
QVQLQESGGGLVQPGGSLRLSCAASGFPFSSYVMIWVRQVPGKGLEWVSAIGGSGGSTYYADSVKGRFTISRDNSKNTLYLQMNSLRADDTAVYYCVLSPKSYYDNSGIYFDFWGKGTLVRV

SEQ ID NO: 22 - protein, 98 aa, Homo Sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 23 - protein, 108 aa, Homo Sapiens
LPELTQPPSVSVAPGKTARITCGGNNIGSKSVYWYQQKPGQAPVLVVYEDSDRPSGLPERFSGSNSGNTATLTISRVEAGDEADYYCQVWDSTSDHWVFGGRTKLTVL

SEQ ID NO: 24 - protein, 96 aa, Homo Sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 25 - protein, 111 aa, Homo Sapiens
QAVLTQPPSASGTPGQRVTISCSGSSSNIGSYTVNWYQQLPGSAPKLLIYSNNHRPSGVPDRFSGSKSGTSASLAISGLQSEDEADYYCAAWADRQNGWVFGGGTKLTVLG

SEQ ID NO: 26 - protein, 98 aa, Homo Sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 27 - protein, 122 aa, Homo Sapiens
EVQLVESGGGLVQPGGSLRLSCAASGFPFSSYVMIWVRQVPGKGLEWVSAIGGSGGSTYYADSVKGRFTISRDNSKNTLYLQMNSLRADDTAVYYCVLSPKSYYDNSGIYFDFWGQGTLVRV

SEQ ID NO: 28 - protein, 112 aa, Homo Sapiens
NFMLTQPHSVSESPGKTITISCTASSGSIASNYVQWYQQRPGSAPTIVIYEDNQRPSGVPDRFSGSIDTSSNSASLTISGLKTEDEADYYCQSYDNTNHWVFGGGTKVTVLG

SEQ ID NO: 29 - protein, 98 aa, Homo Sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSN

SEQ ID NO: 30 - protein, 124 aa, Homo Sapiens
MAEVQLVESGGGLVQPGGSLRLSCAASGFPFSSYVMIWVRQVPGKGLEWVSAIGGSGGSTYYADSVKGRFTISRDNSKNTLYLQMNSLRADDTAVYYCVLSPKSYYDNSGIYFDFWGRGTLVRV

SEQ ID NO: 31 - protein, 112 aa, Homo Sapiens
QSVLTQPPSVSGAPGQRVTISCTGSSSNTGAGNHVHWYQQVAGAAPKLLISNNNNRPSGVPDRFSASKSGTSASLDITGLQAEDEADYYCQSYDNSLNDWVFGGGTKLTVLG

SEQ ID NO: 32 - protein, 99 aa, Homo Sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 33 - protein, 121 aa, Homo Sapiens
QVQLVQSGGGLVQPGGSLRLSCAASGFTLSTYGMHWVRQAAGKGLEWVAVSSYDGRNEYYADSVKGRFTISRDNFKDTLYLQMNNLRAEDTAVYYCAKEVGMRSYDSYGMDVWGKGTLVRV

SEQ ID NO: 34 - protein, 108 aa, Homo Sapiens
DIQLTQSPSTLSASVGGRVTITCRASQSIGPWLAWYQQKPGKAPELLIYEASSLESGVPSRFSGGGSGTEFTLTISRLQPDDIATYYCQQYSSYPWTFGQGTKLEIKR

SEQ ID NO: 35 - protein, 95 aa, Homo Sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 36 - protein, 98 aa, Homo Sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 37 - protein, 121 aa, Homo Sapiens
EVQLVESGGGVVQPGRSLRLSCAASGFTFSSYGMHWVRQAPGKGLEWVAHISYDGTETHYTDSVKGRFTISRDNSMETAYLQMDSLRAEDTAVYYCAKDVSLRAYDHYGMDVWGRGTLVRV

SEQ ID NO: 38 - protein, 109 aa, Homo Sapiens
SSELTQDPAVSVALGQTVRITCQGESLRNYYANWYQQRPGQAPVLVIYGGNSRPSGIADRFSGSSSGITASLTITGAQAEDEADYYCDSRDSSDNHRVFGGRTQLTVLS

SEQ ID NO: 39 - protein, 96 aa, Homo Sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 40 - protein, 121 aa, Homo Sapiens
QVQLVQSGGGVVQPGRSLRLSCGASGFTLSTYGMHWVRQAAGKGLEWVAVSSYDGRNEYYADSVKGRFTISRDNFKDTLYLQMNNLRAEDTAVYYCAKEVGMRSYDSYGMDVWGRGTLVRV

SEQ ID NO: 41 - protein, 108 aa, Homo Sapiens
DIQLTQSPSSVSASVGDRVTITCRASQDISNWLAWYQQKPGEAPKVLIFGASSLQSGVPSRFSGSGSGTEFTLTISSLQAEDFATYYCQQSNNFPYTFGQGTKLEIKR

SEQ ID NO: 42 - protein, 95 aa, Homo Sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 43 - protein, 121 aa, Homo Sapiens
EVQLVESGGGVVQPGRTLRLSCAASGFTFTSYGMHWVRQAPGKGLEWVAVISYDGRKKYYLDSVKGRFTISRDNSMETAYLQMDSLRPEDTAVYYCAKDVSLRAYDHYGMDVWGQGTLVRV

SEQ ID NO: 44 - protein, 108 aa, Homo Sapiens
DIQMTQSPSSLSASVGDRVTITCRASQGISNYLAWYQQKPGKVPKLLIYAASTLQSGVPSRFSGSGSGTDFTLTISSLQPEDVATYFCQQSYSTPFTFGGGTKVDIKR

SEQ ID NO: 45 - protein, 95 aa, Homo Sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 46 - protein, 270 aa, Homo Sapiens
MAQVQLQQSGPGLVKPSETLSLTCSVSGDSIKSRRYYWAWIRQPPGKGMEFIGRLSHDGSTYYTPSLKSRLTISPDTSKNQFSLKLSSVTAADTAMYYCARGVYDWGNSYQLDVWGQGTLVTVSSGGGGSGGGGSGGGGSSALDIQMTQSPSTLSTSVGDRVTITCRASQNISNWLAWYQQKPGKAPKLLIYKASSLESGVPSRFSGSGSGTEFTLTIASLQPDDFATYYCQQYYRSWTFGQGTKLEIKRAAAHHHHHHGEQKLISEEDL

SEQ ID NO: 47 - protein, 269 aa, Homo Sapiens
MAQVQLVQSGAEVKKPGESLKISCKSSGYKLSSYWIAWVRQVPGKGLEWVGIIDPRDSDTRYSPSFEGQVTISADKSINTAYLQWSSLKTADTAMYFCARQADGYRSFYGMDVWGRGTLVTVSSGGGGSGGGGSGGGGSSALDIQLTQSPSSLSASVGDRVTITCRASQTINNYLNWYQQKPGKAPKLLIYAASSLQSGVPSRFSGSGSGTDFTLTISSLQPEDHASYYCQQSYSTPVTFGQGTRLEIKRAAAHHHHHHGEQKLISEDL

SEQ ID NO: 48 - protein, 272 aa, Homo Sapiens
MAQMQLVQSGAEVKKPGSSVKISCKASGGTFNKYIISWVRQAPGQGLEWVGRIVPITGITNYAQRLQGRVTISADKSTNTAYMELRSLKSEDTAVYYCARDQGDLWPHQYQGTDVWGKGTTVTVSSGGGGSGGGGSGGGGSSAEIVLTQSPLSLPVTPGEPASISCRSSQSLLYSNGYNYLDWYLQKPGQSPQLLIYLGSNRASGVPDRFSGSGSGTDFTLKISRVEAEDVGVYYCMQALQVPHTFGQGTKVEIKRAAAHHHHHHGEQKIDL

SEQ ID NO: 49 - protein, 269 aa, Homo Sapiens
MAQVQLQQSGGGVVQPGRSLRLSCTTSGFIFKTYDMHWLRQAPGKGLEWVAFIRHDGRDIKYADSVKGRFTISRDDSKDTLYLQMDSLRAEDTAVYFCARNRFTGYNYFEHWGQGTLVTVSSGGGGSGGGGSGGGGSSALEIVMTQSPGTLSLSPGERATLSCRASQSVSSRYLAWYQQKPGQAPRLLIYGVSSRATGIPDRFSGSGSGTDFTLTISRLEPEDFAVYYCQQYAASPETFGQGTKVEIKRAAAHHHHHHGEQKLISEEDL

SEQ ID NO: 50 - protein, 268 aa, Homo Sapiens
MAEVQLVESGGGLAQPGGSLRLSCSASGFSFKTYDMHWVRQAPGKGPEWISKIDYGNRTTDYADSVKGRFTISRDNAKNSLYLQMSSLRAEDTAVYYCARTRFSGYDYFEDWGKGTLVTVSSGGGGSGGGGSGGGGSSALEIVLTQSPGTLSLSPGESATLSCRPSQSVSSRDLAWYQQKPGQAPRLLIYGASSRATGIPDRFSGSGSGTEFTLTITRLEPEDFAVYYCQQYGRSPLTFGGGTKVEIKRAAAHHHHHHGEQKLISEDL

SEQ ID NO: 51 - protein, 265 aa, Homo Sapiens
MAEVQLVESGGGVVQPGRSLRLSCAASGFSFSTYDMHWVRRAPGKGLEWVAHIRFDGSKTSYADSVKGRFTISRDNTKNTLFLQMNSLRGEDTAIYYCARVRFSGYDYFENWGKGTLVTVSSGGGGSGGGGSGGGGSVHLKLTQSPATLSVSPGESATLSCRASQSVRSYLAWYQQKPGQAPRLLIYGASSRATGIPDRFSGSGSGTDFTLTISRLEPEDFAVYYCQQYGSSPVTFGQGTKLEIKRAAAHHHHHHGEQKLISEDL

SEQ ID NO: 52 - protein, 267 aa, Homo Sapiens
MAEVQLVESGGGVVQSGGSLRLSCAASGFTFSGYDMHWVRQAPGKGLEWVAGLRYDGTKREYADSVRGRFTISRDNTKNTLYLQMDSLRGEDTAVYFCARVRFSGYNYFENWGKGTTVTVSSGGGGSGGGGSGGGGSSALEIVMTQSPGTLSLSPGERATLSCRASQSVSSRYLAWYQQKPGQAPRLLIYGVSSRATGIPDRFSGSGSGTDFTLTISRLEPEDFAVYYCQQYAASPETFGQGTKVEIKRAAAHHHHHHGEQKLISNL

SEQ ID NO: 53 - protein, 269 aa, Homo Sapiens
MAEVQLVESGGGLVKPGGSLRLSCAASGFSFSTYDMHWVRQAPGKGLEWVAGLRYDGSKKYYADSVKGRFTISRDNTKNTLYLQMDSLRGEDTAVYYCARVRFSGYEYFENWGQGTLVTVSSGGGGSGGGGSGGGGSSALDVVMTQSPSTLSASVGDRVTITCRASQSVSTWLAWYQQKPGKAPKLLIYQASNLESGVPSRFSGSGSDTEFTLTINNLQPADFATYYCQQYNTYSSATFGQGTKVEIKRAAAHHHHHHGEQKLISEEDL

SEQ ID NO: 54 - protein, 269 aa, Homo Sapiens
MAEVQLVESGGGVVQPGRSLRLSCAASGFSFSTYDMHWVRQAPGKGLEWVAHIRFDGSKTSYADSVKGRFTISRDNTKNTLFLQMNSLRGEDTAIYYCARVRFSGYDYFENWGQGTLVTVSSGGGGSGGGGSGGGGSSALEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKLEIKRAAAHHHHHHGEQKLISEDL

SEQ ID NO: 55 - protein, 267 aa, Homo Sapiens
MAEVQLVESGGNVVQPGRSLRLSCAASGLSFSTYDMHWVRQAPGKGLEWVAGVRYDGSKKYYTESVRGRFTISRDNTKNTLFLQMDSLRGDDTAVYYCARVRFSGYDYFENWGKGTLVTVSSGGGGSGGGGSGGGGSSALEIVMTQSPATLSVSPGERATLSCRASQSVSSNLAWYQQKPGQAPRLLIYGASTRATGIPARFSGSGSGTEFTLTISGLQSEDFAVYYCQQYDNWPLTFGGGTKVEIKRAAAHHHHHHGEQKLISEDL

SEQ ID NO: 56 - protein, 268 aa, Homo Sapiens
MAEVQLVESGGGVVQPGRSLRLSCAASGFSFSTYDMHWVRQAPGKGLEWVAGLRYDGSKKYYADSVKGRFTISRDNTKNTLYLQMDSLRGEDTAVYYCARVRFSGYEYFENWGQGTLVTVSSGGGGSGGGGSGGGGSSALEIVMTQSPGTLSLSPGERATLSCRASQSVSSSYLAWYQHKPGQAPRLLIYGASNRATGIPDRFSGSGSGTDFTLTISRLEPEDFAVYYCQQYGSSPFTFGGGTKVEIKRAAAHHHHHHGEQKLISEDL

SEQ ID NO: 57 - protein, 268 aa, Homo Sapiens
MAEVQLVESGGGVVQPGRSLRLSCAASGFSFSTYDMHWVRQAPGKGLEWVAGLRYDGSKKYYADSVKGRFTISRDNTKNTLYLQMDSLRGEDTAVYYCARVRFSGYEYFENWGRGTLVTVSSGGGGSGGGGSGGGGSSALEIVLTQSPGTLSLSPGERVTLSCRASQSVSSTYLAWYQQKPGQAPRLLIYGASSRATGIPDRFSGSGSGTDFTLTVTRLEPEDFAVYYCQQYASSPLTFGGGTKVEIKRAAAHHHHHHGEQKLISEDL

SEQ ID NO: 58 - protein, 269 aa, Homo Sapiens
MAEVQLVETGGGVVQPGRSLRLSCAASGLSFSTYDMHWVRQAPGKGLEWVAGIRYDGSKKYYADSVKGRFTISRDNTRNTLYLQMDSLRGEDTAIYYCARVRFSGYEYFENWGRGTLVTVSSGGGGSGGGGSGGGGSSALEIVMTQSPSTLSLSPGERATLSCRASQSVSSSYLAWYQQKPGQAPRLLIYGASSRATGIPDRFSGSGSGTDFTLTISRLEPEDFAVYYCQQYGSSSYTFGQGTKLEIKRAAAHHHHHHGEQKLISEEDL

SEQ ID NO: 59 - protein, 269 aa, Homo Sapiens
MAEVQLVESGGGLVNPGGSLRLSCAASGFSFSTYDMHWVRQAPGKGLEWVAHIRFDGSKTSYADSVKGRFTISRDNTKNTLFLQMNSLRGEDTAIYYCARVRFSGYDYFENWGKGTLVTVSSGGGGSGGGGSGGGGSSALEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPGTFGQGTKVEIKRAAAHHHHHHGEQKLISEEDL

SEQ ID NO: 60 - protein, 269 aa, Homo Sapiens
LAEVQLVESGGGVVQPGRSLRLSCAASGFSFSTYDMHWVRQAPGKGLEWVAHIRFDGSKTSYADSVKGRFTISRDNTKNTLFLQMNSLRAEDTAIYYCARVRFSGYDYFENWGKGTLVTVSSGGGGSGGGGSGGGGSSALEIVLTQSPGTLSLSPGERATLSCRASQSVSSSYLAWYQQKPGQAPRLLIYGASSRATGIPDRFSGSGSGTDFTLTISRLEPEDFAVYYCQQYGSSLALTFGGGTKVEIKRAAAHHHHHHGEQKLISEDL

SEQ ID NO: 61 - protein, 268 aa, Homo Sapiens
MAQVQLVESGGGVVQPGRSLRLSCAASGFSFSTYDMHWVRQAPGKGLEWVAHIRFDGSKTSYADSVKGRFTISRDNTKNTLFLQMNSLRGEDTAIYYCARVRFSGYDYFENWGKGTLVTVSSGGGGSGGGGSGGGGSSALDIQLTQSPSSVSASVGDRVTITCRASQGISSGLAWYQQNPGKAPNLLIYAASNLQSGVPSRFSGSGSGTDFTLTISSLQPEDFATYYCQQTNSFPLTFGGGTKVEIKRAAAHHHHHHGEQKLISEEDL

SEQ ID NO: 62 - protein, 268 aa, Homo Sapiens
MAQVQLVESGGGVVQPGRSLRLSCAASGFSFSTYDMHWVRQAPGKGLEWVAHIRFDGSKTSYADSVKGRFTISRDNTKNTLFLQMNSLRGEDTAIYYCARVRFSGYDYFENWGRGTLVTVSSGGGGSGGGGSGGGGSSALEIVMTQSPGTLSLSPGERATLSCRASQSVSSRYLAWYQQKPGQAPRLLIYGVSSRATGIPDRFSGSGSGTDFTLTISRLEPEDFAVYYCQQYAASPETFGQGTKVEIKRAAAHHHHHHGEQKLISEDL

SEQ ID NO: 63 - protein, 268 aa, Homo Sapiens
MAEVQLVESGGGVVQPGRSLRLSCAASGLSFSTYDMHWVRQAPGKGLEWVAGIRYDGSKKYYADSVKGRFTISRDNTRNTLYLQMDSLRGEDTAIYYCARVRFSGYEYFENWGKGTMVTVSSGGGGSGGGGSGGGGSSALEIVMTQSPGTLSLSPGERATLSCRASQSVSSRYLAWYQQKPGQAPRLLIYGVSSRATGIPDRFSGSGSGTDFTLTISRLEPEDFAVYYCQQYAASPETFGQGTKVEIKRAAAHHHHHHGEQKLISEDL

SEQ ID NO: 64 - protein, 268 aa, Homo Sapiens
MAQVQLVESGGGLVQPGGSLRLSCAASGLSFPTYDMHWVRQAPGKGLEWVAHIRFDGTKTSYGDAVRGRFTISRDNTKNTLYLQMNRLRGEDTAIYYCARVRFSGYDYFENWGRGTTVTVSSGGGGSGGGGSGGGGSSALEIVMTQSPATLSVSPGERATLSCRASQSVSSNLAWYQQKPGQAPRLLIYGASTRATGIPARFSGSGSGTEFTLTISGLQSEDFAVYYCQQYDNWPLTFGGGTKVEIKRAAAHHHHHHGEQKLISEEDL

SEQ ID NO: 65 - protein, 270 aa, Homo Sapiens
MAEVQLVETGGGVVQPGRSLRLSCAASGFSFSTYDMHWVRQAPGKGLEWVAGLRYDGSKKYYADSVKGRFTISRDNTKNTLYLQMDSLRGEDTAVYYCARVRFSGYEYFENWGKGTLVTVSSGGGGSGGGGSGGGGSSAQSALTQPRSVSGSPGQSVTISCTGTSSDVGGYNYVSWYQQHPGKAPKLMIYDVSNRPSGVSNRFSGSKSGNTASLTISGLQAEDEADYYCSSYTSSSTLVIFGGRTKLTVLGAAAHHHHHHGEQKLISENC

SEQ ID NO: 66 - protein, 276 aa, Homo Sapiens
MAEVQLVQSGAEVKKPGSSVKVSCKASGVTLSIYSMNWVRQAPGQGLEWMGRIIPITGVPNYSQNFQGRVTITADKSTSTTYMELSSLRSEDTAVYYCALSGAGYNYYGMDVWGQGTLVTVSSGGGGSGGGGSGGGALEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRAAAHHHHHHGEQKLISENCKLLKVV

SEQ ID NO: 67 - protein, 272 aa, Homo Sapiens
MAEVQLVQSGAEVKKPGSSVKVSCKASGVTLSIYSMNWVRQAPGQGLEWMGRIIPITGVPNYSQNFQGRVTITADKSTSTTYMELSSLRSEDTAVYYCALSGAGYNYYGMDVWGKGTLVTVSSGGGGSGGGGSGGGGSSALEIVLTQSPLSLPVTPGEPASISCRSSQSLLHSNGYNYLDWYLQKPGQSPHLLIYLGSNRASGVPDRFSGSGSGTDFTLKISRVEAEDVGVYYCMQALQTPRTFGPGTKVEIKRAAAHHHHHHGEQKLISDL

SEQ ID NO: 68 - protein, 270 aa, Homo Sapiens
MAEVQLVESGGGLVQPGRSLRLSCGASGFTLSTYGMHWVRQAAGKGLEWVAVSSYDGRNEYYADSVKGRFTISRDNFKDTLYLQMNNLRAEDTAVYYCAKEVGMRSYDSYGMDVWGQGTMVTVSSGGGGSGGGGSGGGGSSALDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKRAAAHHHHHHGEQKLISEDL

SEQ ID NO: 69 - protein, 264 aa, Homo Sapiens
MAQVQLQQSGGGLVQPGGSLRLSCTASGFTFSNYHMNWVRQAPGKGLEWVSHISSSSRTIKYADSVKGRFTISRDNAKNSLYLQMNSLRAEDTAVYYCARAGSGYSSGPTDYWGKGTMVTVSSGGGGSGGGGSGGGGSSAQSVLTQLPSVSGAPGQRVTISCTGSSSNIGAGYDVHWYQQLPGTAPKLLIYGNSNRPSGVPDRFSGSKSGTSASLAITGLQADADYYCQSYDTNLRAYVFGTGTKLTVLAAAHHHHHHGKQNSQ

SEQ ID NO: 70 - protein, 275 aa, Homo Sapiens
MAEVQLVESGGGVVQPGRSLRLSCTASGFSSSVYDMHWVRQAPGQGLEWVALISHDGNHKHYADSVKGRFTISRDNSKNALYLQMDSLRGEDTAVYYCARDRFGRSGIKLKVTYLDYWGEGTTVTVSSGGGGSGGGGSGGGGSSALEIVLTQSPASLSLSPGERATLSCRASESIHRYLAWYQQKPGQAPRLLIYDTSNRATGIPARFSGSGSGTDFTLTISSLEPEDFAVYYCQQYNSWPPITFGQGTRLEIKRAAAHHHHHHGEQKLISEEDL

SEQ ID NO: 71 - protein, 268 aa, Homo Sapiens
MAQVQLVESGGGVVQPGKSLRLSCAASGFDFSGYGMHWVRQAPGKGLEWVAFISYDASNQYYADSVKGRFTVSRDNSKNTVSLQMSSLKTDDTAVYYCAKDFSWSGSIDSWGQGTLVTVSSGGGGSGGGGSGGGGSSALDVVMTQSPGTLSLSPGERATLSCRASQSVSSSYLAWYQQKPGQAPRLLIYGASSRATGIPDRFSGSGSGTDFTLTISRLEPEDFAVYYCQQYGSSLSLTFGGGTKVEIKRAAAHHHHHHGEQKLISEEL

SEQ ID NO: 72 - protein, 273 aa, Homo Sapiens
MAEVQLVESGGGVVQPGRSLRLSCTASGFSSSVYDMHWVRQAPGQGLEWVALISHDGNHKHYADSVKGRFTISRDNSKNALYLQMDSLRGEDTAVYYCARDRFGRSGIKLKVTYLDYWGKGTLVTVSSGGGGSGGGGSGGGGSSALDIQMTQSPSSLSASVGDRVTITCRASQNISSFLNWYQQKPGKAPKLLIYATSRLQSGVPSRFSGSGSGTDFTLTISSLQPEDFATYYCQQSYNTPLTFGGGTKVDIKRAAAHHHHHHGEQKLISEDL

SEQ ID NO: 73 - protein, 273 aa, Homo Sapiens
MAQVQLVESGGGVVQPGRSLRLSCAASGFTSSPYDMHWVRQAPGQGLEWVALISHDGSYKHYTDSVKGRFTISRDNSKNALYLQMNSLRAEDTAVYYCARDRFGRSGIKLKVTYLDYWGKGTLVTVSSGGGGSGGGGSGGGGSSALEIVLTQSPGTLSLSPGERATLSCRASESVDNTFLAWYQQKPGQAPRLLIYGASSRATGIPDRFSGGGSGTDFTLTISRLEPEDSAVYYCQQYGNSLNFGGGTKLEIKRAAAHHHHHHGEQKLISEDL

SEQ ID NO: 74 - protein, 273 aa, Homo Sapiens
MAEVQLVESGGGVVQPGRSLRLSCTASGFSSSVYDMHWVRQAPGQGLEWVALISHDGNHKHYADSVKGRFTISRDNSKNALYLQMDSLRGEDTAVYYCARDRFGRSGIKLKVTYLDYWGRGTLVTVSSGGGGSGGGGSGGGGSSALDIQLTQSPSTLSASVGDRVTITCRASQSITNLLAWYQQKPGKAPNLLIYKTSTLQSGVPSRFSGSGSGTEFTLTISSLQPDDFATYYCQQYHRFSYSFGQGTKVEIKRAAAHHHHHHGEQKLISENL

SEQ ID NO: 75 - protein, 273 aa, Homo Sapiens
MAEVQLVESGGGVVQPGRSLRLSCAASGFSSSVYDMHWVRQAPGQGLEWVALISHDGNHKHYTDSVKGRFTISRDNSKNALYLQMNSLRGEDTAVYYCARDRFGRSGMKLKVTYLDYWGKGTLVTVSSGGGGSGGGGSGGGGSSALDIQMTQSPSSLSASVGDRVTLTCRASQSINAYLNWYQQKPGKAPKLLIYTASSLQSGVPSRFSGSGSGTDFTLTISRLQPEDFATYYCQQTYSSPLTFGGGTKVEIKRAAAHHHHHHGEQKLISEDL

SEQ ID NO: 76 - protein, 276 aa, Homo Sapiens
MAEVQLVETGGGVVQPGRSLRLSCTASGFSSSVYDMHWVRQAPGQGLEWVALISHDGNHKHYADSVKGRFTISRDNSKNALYLQMDSLRGEDTAVYYCARDRFGRSGIKLKVTYLDYWGRGTTVTVSSGGGGSGGGGSGGGGSSAQSVLTQPPSVSAAPGQKVTISCSGSSSNIGKNYVSWYQQVPGTAPKLLIYDNNKRPSGIPDRFSGSKSGTSATLGITGLQTGDEADYHCGTWDSSLHSGLFGGGTKLTVLGAAAHHHHHHGEQKLISEEDL

SEQ ID NO: 77 - protein, 268 aa, Homo Sapiens
MAEVQLVESGGDLVRPGGSLRLSCTISGVTFNQYAITWVRQAPGKGLQWLSTIAGTGTKTFYADSVKGRFTMSRDSSGNTLYLQMNSLRDEDTAVYYCAKSLSMRYFLDLWGRGTLVTVSSGGGGSGGGGSGGGGSSAHVILTQPPSASGTPGQRVIISCSGSSSNIGSNTVNWYQQLPGTAPKLLIYNNNQRPSGVPDRFSGSKSGTSASLAISGLQSEDEADYYCAAWDDSLNGWVFGGGTKVTVLAAAHHHHHHGEQKLISEEDL

SEQ ID NO: 78 - protein, 271 aa, Homo Sapiens
MAQVQLQESGGDLVKPGGSLRLSCVASGLTFNSYGMSWVRQAPGKGLEWVSDISASGFNTYYVDSLKGRFTISRDNSRNTLFLQMNNLRDEDTAIYYCAKNGGDYMGAYIDNWGKGTLVTVSSGGGGSGGGGSGGGGSSAQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTQLTVLGAAAHHHHHHGEQKLISEEDL

SEQ ID NO: 79 - protein, 269 aa, Homo Sapiens
MAQVQLVQSGAEVKKPGSSVKVSCRASGGTFRSYSFNWLRQAPGQGLEWMGRIIPVVGVLDYAPKFQARVTFTVDTSTSVGYMDLNGLTPEDTAVYYCAGGDHVVKAALAYWGGGTTVTVSSGGGGSGGGGSGGGGSSAQSALTQPASESGSPGQSITISCTGTSTDVGARNSVSWYQQHPGKAPKLILYDVSHRPSGVSNRFSGSKSGNTASLTISGLQAEDEGDFYCSSYTTSNNLVFGGGTKLTVLGAAAHHHHHHGEQKLISEDL

SEQ ID NO: 80 - protein, 269 aa, Homo Sapiens
MAQVQLQQSGPGLVKPSETLSLTCTLSGGSMESHYWSWIRQPPGKGLEWMGRVSYIGISNYNPYLKNRVTISQDKSKNQLSLRLNSVTAADTAVYYCARHRLRSDQAFDLWGKGTLVTVSSGGGGSGGGGSGGGGSSAQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTQLTVLSAAAHHHHHHGEQKLISEDL

SEQ ID NO: 81 - protein, 269 aa, Homo Sapiens
MAEVQLVESGGGVVQPGRSLRLSCTISGVTFNQYAITWVRQAPGKGLQWLSTIAGTGTKTFYADSVKGRFTMSRDSSGNTLYLQMNSLRDEDTAVYYCAKSLSMRYFLDLWGRGTLVTVSSGGGGSGGGGSGGGGSSALPELTQPPSASGTPGQRVTISCSGSSSNIGSNTVNWYQQLPGTAPKLLIYSNNQRPSGVPDRFSGSKSGTSASLAISGLQSEDEADYYCAAWDDSLNGLVFGGGTKLTVLGAAAHHHHHHGEQKLISEEDL

SEQ ID NO: 82 - protein, 268 aa, Homo Sapiens
MAEVQLVETGGDLVRPGGSLRLSCTISGVTFNQYAITWVRQAPGKGLQWLSTIAGTGTKTFYADSVKGRFTMSRDSSGNTLYLQMNSLRDEDTAVYYCAKSLSMRYFLDLWGQGTMVTVSSGGGGSGGGGSGGGGSSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVLAAAHHHHHHGEQKLISEEDL

SEQ ID NO: 83 - protein, 270 aa, Homo Sapiens
MAQVQLQESGPGLVKPSETLSLTCSVSGDAISNGYFWGWIRQPPGGGLEWIGSISHRGSTYYNPSLKSRVSISVDTSKNQFSLSLTSVTAADTAVFYCARSNGDYDTFTAYYWGRGTMVTVSSGGGGSGGGGSGGGGSSAQAVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKVTVLAAAHHHHHHGEQKLISEDL

SEQ ID NO: 84 - protein, 269 aa, Homo Sapiens
MAQVQLQESGPGLLKPSQTLSLTCTVSGDSISSGDHYWNWIRQPAGKGLEWIGRLYTNGITDYNPSLRSRVIISADTSKNQFTLKLSAVTAADTAVYYCARDVWEPGTFEHWGKGTMVTVSSGGGGSGGGGSGGGGSSALSSELTQDPAVSVALGQTVRITCQGDSLRSYYASWYQQKPGQAPILVIYGKNNRPSGIPDRFSGSSSGNTASLTITGAQAEDEADYYCNSRDSNGDVLSVFGGGTKLTVLAAAHHHHHHGEQKLISEEDL

(19) United States
(12) Patent Application Publication
(10) Pub. No.: US 2008/0014205 A1
(43) Pub. Date: Jan. 17, 2008

(54) Neutralizing Antibodies to Influenza Viruses

(76) Inventors: Lawrence Horowitz, Atherton, CA (US); Ramesh Bhatt, Belmont, CA (US); Arun K. Kashyap, Newark, CA (US)

Correspondence Address: HELLER EHRMAN LLP, 275 MIDDLEFIELD ROAD, MENLO PARK, CA, 94025-3506, US

(21) Appl. No.: 11/748,980

(22) Filed: May 15, 2007

Related U.S. Application Data
(60) Provisional application No. 60/800,787, filed on May 15, 2006.
(60) Provisional application No. 60/855,679, filed on Oct. 30, 2006.

(51) Int. Cl. A61K 39/395; C07K 16/08; C40B 30/04; C40B 40/10

(52) U.S. Cl. 424/159.1; 506/18; 506/9; 530/387.3; 530/389.4

(57) ABSTRACT

The present invention concerns methods and means for identifying, producing, and engineering neutralizing antibodies against influenza A viruses, and to the neutralizing antibodies produced. In particular, the invention concerns neutralizing antibodies against various influenza A virus subtypes, including neutralizing antibodies against two or more of H1, H2, H3, H5, H7 and H9, such as, for example all of H1, H2, H3, and H5 subtypes, and methods and means for making such antibodies. More specifically, the invention concerns antibodies capable of neutralizing more than one, preferably all, isolates of an influenza A virus subtype.

### CROSS REFERENCE TO RELATED APPLICATION

This application claims priority under 35 U.S.C. §119(e) from U.S. provisional patent application Nos. 60/800,787, filed May 15, 2006 and 60/855,679, filed Oct. 30, 2006, the entire contents of which are incorporated by reference.

### FIELD OF THE INVENTION

The present invention concerns methods and means for identifying, producing, and engineering neutralizing antibodies against influenza A viruses, and to the neutralizing antibodies produced. In particular, the invention concerns neutralizing antibodies against various influenza A virus subtypes, including neutralizing antibodies against two or more of H1, H2, H3, H5, H7 and H9, such as, for example all of H1, H2, H3, and H5 subtypes, and methods and means for making such antibodies. More specifically, the invention concerns antibodies capable of neutralizing more than one, preferably all, isolates of an influenza A virus subtype.

### BACKGROUND OF THE INVENTION

The flu is a contagious respiratory illness caused by influenza viruses. It causes mild to severe illness, and at times can lead to death. Annually, in the United States, influenza is contracted by 5-20% of the population, hospitalizing about 200,000, and causing the deaths of about 36,000.

Influenza viruses spread in respiratory droplets caused by coughing and sneezing, which are usually transmitted from person to person. Immunity to influenza surface antigens, particularly hemagglutinin, reduces the likelihood of infection and severity of disease if infection occurs. Although influenza vaccines are available, because an antibody against one influenza virus type or subtype confers limited or no protection against another type or subtype of influenza, it is necessary to incorporate one or more new strains in each year's influenza vaccine.

Influenza viruses are segmented negative-strand RNA viruses and belong to the Orthomyxoviridae family. Influenza A virus consists of 9 structural proteins and codes additionally for one nonstructural NS1 protein with regulatory functions. The non-structural NS1 protein is synthesized in large quantities during the reproduction cycle and is localized in the cytosol and nucleus of the infected cells. The segmented nature of the viral genome allows the mechanism of genetic reassortment (exchange of genome segments) to take place during mixed infection of a cell with different viral strains. The influenza A virus is further classified into various subtypes depending on the different hemagglutinin (HA) and neuraminidase (NA) viral proteins displayed on their surface.

Influenza A virus subtypes are identified by two viral surface glycoproteins, hemagglutinin (HA or H) and neuraminidase (NA or N). Each influenza virus subtype is identified by its combination of H and N proteins. There are 16 known HA subtypes and 9 known NA subtypes. Influenza type A viruses can infect people, birds, pigs, horses, and other animals, but wild birds are the natural hosts for these viruses. Only some influenza A subtypes (i.e., H1N1, H1N2, and H3N2) are currently in circulation among people, but all combinations of the 16 H and 9 NA subtypes have been identified in avian species, especially in wild waterfowl and shorebirds. In addition, there is increasing evidence that H5 and H7 influenza viruses can also cause human illness.

The HA of influenza A virus comprises two structurally distinct regions, namely, a globular head region and a stem region. The globular head region contains a receptor binding site which is responsible for virus attachment to a target cell and participates in the hemagglutination activity of HA. The stem region contains a fusion peptide which is necessary for membrane fusion between the viral envelope and an endosomal membrane of the cell and thus relates to fusion activity (Wiley et al., Ann. Rev. Biochem., 56:365-394 (1987)).

A pandemic is a global disease outbreak. An influenza pandemic occurs when a new influenza A virus: (1) emerges for which there is little or no immunity in the human population, (2) begins to cause serious illness, and then (3) spreads easily person-to-person worldwide. During the 20^{th} century there have been three such influenza pandemics. First, in 1918, the “Spanish Flu” influenza pandemic caused at least 500,000 deaths in the United States and up to 40 million deaths worldwide. This pandemic was caused by influenza A H1N1 subtype. Second, in 1957, the “Asian Flu” influenza pandemic, caused by the influenza A H2N2 subtype, resulted in at least 70,000 deaths in the United States and 1-2 million deaths worldwide. Most recently in 1968 the “Hong Kong Flu” influenza pandemic, caused by the influenza A H3N2 subtype, resulted in about 34,000 U.S. deaths and 700,000 deaths worldwide.

In 1997, the first influenza A H5N1 cases were reported in Hong Kong. This was the first time that this avian type virus directly infected humans, but a pandemic did not result because human to human transmission was not observed.

Lu et al., Resp. Res. 7:43 (2006) (doi: 10.1186/1465-992-7-43) report the preparation of anti-H51 IgGs from horses vaccinated with inactivated H5N1 virus, and of H5N 1-specific F(ab′)₂ fragments, which were described to protect BALB/c mice infected with H5N1 virus.

Hanson et al., Resp. Res. 7:126 (doi: 10.1186/1465-9921-7-126) describe the use of a chimeric monoclonal antibody specific for influenza A H5 virus hemagglutinin for passive immunization of mice.

In view of the severity of the respiratory illness caused by certain influenza A viruses, and the threat of a potential pandemic, there is a great need for effective preventative and treatment methods. The present invention addresses this need by providing influenza A neutralizing antibodies against various H subtypes of the virus, including, without limitation, the H1, and H3 subtypes, and the H5 subtype of the influenza A virus. The invention further provides antibodies capable of neutralizing more than one, and preferably all, isolates (strains) of a given subtype of the influenza A virus, including, without limitation, isolates obtained from various human and non-human species and isolates from victims and/or survivors of various influenza epidemics and/or pandemics.

Such neutralizing antibodies can be used for the prevention and/or treatment influenza virus infection, including passive immunization of infected or at risk populations in cases of epidemics or pandemics.

### SUMMARY OF THE INVENTION

In one aspect, the present invention concerns a neutralizing antibody neutralizing more than one isolate of an influenza A virus subtype or more than one subtype of the influenza A virus.

In one embodiment, the antibody neutralizes substantially all isolates of an influenza A virus subtype, such as one or more of the H5, H7 and H9 subtypes.

In another embodiment, the antibody neutralizes more than one isolate of a particular influenza A virus subtype, such as one or more of the H5, H7 and H9 subtypes.

In yet another embodiment, the antibody neutralizes more than one subtype and more than one isolates of at least one subtype of the influenza A virus.

In a further embodiment, at least one of the subtypes and/or isolates neutralized by the antibodies herein has the ability to infect humans.

In another embodiment, at least one of the isolates is from a bird, including, for example, wild-fowls and chicken.

In a particular embodiment the antibodies herein neutralize the H5N1 subtype of the influenza A virus. Preferably, the antibodies neutralize more than one isolate, or, even more preferably, substantially all isolates of this influenza A virus subtype.

In another embodiment, the antibodies herein neutralize the H5N1 subtype and at least one additional subtype selected from the group consisting of H1N1, H1N2, and H3N2 subtypes.

In additional embodiments, the antibodies herein neutralize more than one isolate, preferably substantially all isolates of the additional subtype(s).

In another embodiment, the neutralizing antibodies of the present invention bind the H5 protein. Preferably, the antibodies bind more than one variants of the H5 protein, or, even more preferably, substantially all variants of the H5 protein.

In other embodiments, the antibodies herein bind to the H5 protein and to at least one additional H protein, such as an H1, H2 and/or H3 protein.

In a different aspect, the invention concerns compositions comprising the neutralizing antibodies described herein.

In a further aspect, the invention concerns a method for identifying an antibody capable of neutralizing more than one isolate of a single influenza A virus subtype or multiple influenza A virus subtypes. This method comprises identifying antibodies in an antibody library that react with both a first and a second isolate of the influenza A virus subtype or with a first and a second subtype of the influenza A virus, and subjecting the antibodies identified to successive alternating rounds of selection, based on their ability to bind the first and second isolates, or the first and second subtypes, respectively.

In an embodiment, antibodies that react with both a first and a second influenza A virus subtype isolate have been identified by at least two rounds of separate enrichment of antibodies reacting with the first isolate and the second isolate, respectively, and recombining the antibodies identified.

In another embodiment, the antibody that can react with both the first and the second influenza A subtype isolate is subjected to mutagenesis prior to being subjected to successive alternating rounds of selection, based on its ability to bind the first and second isolate, respectively. If desired, the antibodies capable of binding the first and the second isolate are additionally selected based on their ability to bind more than one influenza A subtype.

The application of such enrichment techniques can be similarly applied to antibodies in general, regardless of the target to which they bind. Such general enrichment/selection methods are specifically included as part of the invention.

In a further aspect, the invention concerns a collection of sequences shared by the neutralizing antibodies of the present invention.

In a still further aspect, the invention concerns a method for treating an influenza A infection in a subject comprising of administering to the subject an effective amount of a neutralizing antibody or antibody composition herein.

In another aspect, the invention concerns a method for preventing influenza A infection comprising of administering to a subject at risk of developing influenza A infection an effective amount of a neutralizing antibody of the present invention.

In a different aspect, the invention concerns a method for producing a diverse multifunctional antibody collection, comprising: (a) aligning CDR sequences of at least two functionally different antibodies, (b) identifying amino acid residues conserved between the CDR sequences aligned, and (c) performing mutagenesis of multiple non-conserved amino acid residues in at least one of the CDR sequences aligned, using degenerate oligonucleotide probes encoding at least the amino acid residues present in the functionally different antibodies at the non-conserved positions mutagenized to produce multiple variants of the aligned CDR sequences, and, if desired, repeating steps (b) and (c) with one or more of the variants until the antibody collection reaches a desired degree of diversity and/or size.

In a particular embodiment, the CDR sequences aligned have the same lengths.

In another embodiment, the conserved amino acid residues are retained in at least two of the CDR sequences aligned.

In a further aspect, the invention concerns an antibody collection comprising a plurality of neutralizing antibodies which differ from each other in at least one property.

The invention further concerns a method for uniquely identifying nucleic acids in a collection comprising labeling the nucleic acids with a unique barcode linked to or incorporated in the sequences of the nucleic acid present in such collection.

### BRIEF DESCRIPTION OF THE DRAWINGS

FIG. 1 shows the amino acid sequences of 15 known hemagglutinin (H) protein subtypes.

FIG. 2 illustrates a typical panning enrichment scheme for increasing the reactive strength towards two different targets, A and B. Each round of enrichment increases the reactive strength of the pool towards the individual target(s).

FIG. 3 illustrates a strategy for the selection of clones cross-reactive with targets A and B, in which each successive round reinforces the reactive strength of the resulting pool towards both targets.

FIG. 4 illustrates a strategy for increasing the reactive strengths towards two different targets (targets A and B), by recombining parallel discovery pools to generate/increase cross-reactivity. Each round of selection of the recombined antibody library increases the reactive strength of the resulting pool towards both targets.

FIG. 5 illustrates a strategy for increasing cross-reactivity to a target B while maintaining reactivity to a target A. First, a clone reactive with target A is selected, then a mutagenic library of the clones reactive with target A is prepared, and selection is performed as shown, yielding one or more antibody clones that show strong reactivity with both target A and target B.

FIG. 6 illustrates a representative mutagenesis method for generating a diverse multifunctional antibody collection by the “destinational mutagenesis” method.

FIG. 7 shows the H5 hemagglutinin (HA) serology results for blood samples obtained from six human survivors of a Turkish H5N1 bird flu outbreak. The data demonstrate the presence of antibodies to the HA antigen.

FIG. 8 shows serology results obtained with serum samples of twelve local donors, tested on H5 antigen (A/Vietnam/1203/2004) and H1N1 (A/New Caledonia/20/99) and H3N2 (A/Panama/2007/99) viruses.

FIG. 9 illustrates the unique barcoding approach used in the construction of antibody phage libraries.

FIG. 10 shows the results of a scFv ELISA test of five distinct clones obtained from pooled libraries of Turkish bird flu survivors on H5 protein and H5N1 virus.

FIG. 11 shows sequence alignments comparing the sequences of H5 hemagglutinin proteins from reported Turkish isolates and one Vietnamese isolate downloaded from the Los Alamos National Laboratory sequence database.

FIGS. 12 and 13 show heavy chain variable region sequences of unique clones identified in pooled antibody libraries of Turkish donors, along with the corresponding light chain and germline origin sequences. The sequences shown in FIG. 12 (3-23 heavy chain clones) originate from a pooled library of all heavy and light chains of all Turkish donors after three rounds of panning. The sequences shown in FIG. 13 (3-30 heavy chain clones) originate from a pooled library of all heavy and light chains of all Turkish donors after two rounds of panning.

FIGS. 14A-D show additional unique H5N1-specific antibody heavy chain variable region sequences identified from antibody libraries of individual Turkish donors, after four rounds of panning.

FIGS. 15 and 16 illustrate the use of destinational mutagenesis to create diverse antibody heavy and light chain libraries using the antibody heavy (FIG. 15) and light chain (FIG. 16) sequences identified by analysis of sera and bone marrow of Turkish bird flu survivors.

FIGS. 17 and 18 show ELISA results confirming cross-reactivity of certain Fab fragments obtained from an H5N1 Vietnam virus scFv antibody with Turkish and Indonesian variants of the HA protein.

### DETAILED DESCRIPTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “influenza A subtype” or “influenza A virus subtype” are used interchangeably, and refer to influenza A virus variants that are characterized by various combinations of the hemagglutinin (H) and neuraminidase (N) viral surface proteins, and thus are labeled by a combination of an H number and an N number, such as, for example, H1N1 and H3N2. The terms specifically include all strains (including extinct strains) within each subtype, which usually result from mutations and show different pathogenic profiles. Such strains will also be referred to as various “isolates” of a viral subtype, including all past, present and future isolates. Accordingly, in this context, the terms “strain” and “isolate” are used interchangeably.

The term “influenza” is used to refer to a contagious disease caused by an influenza virus.

In the context of the present invention, the term “antibody” (Ab) is used in the broadest sense and includes polypeptides which exhibit binding specificity to a specific antigen as well as immunoglobulins and other antibody-like molecules which lack antigen specificity. Polypeptides of the latter kind are, for example, produced at low levels by the lymph system and, at increased levels, by myelomas. In the present application, the term “antibody” specifically covers, without limitation, monoclonal antibodies, polyclonal antibodies, and antibody fragments.

“Native antibodies” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al,. J Mol Biol. 1996. “Framework” or “FR” residues are those variable domain residues other than the hypervariable region residues as herein defined.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

“Antibody fragments” comprise a portion of a full length antibody, generally the antigen binding or variable domain thereof. Examples of antibody fragments include, but are not limited to, Fab, Fab′, F(ab′)₂, and Fv fragments, linear antibodies, single-chain antibody molecules, diabodies, and multispecific antibodies formed from antibody fragments.

The term “monoclonal antibody” is used to refer to an antibody molecule synthesized by a single clone of B cells. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage antibody libraries.

The term “polyclonal antibody” is used to refer to a population of antibody molecules synthesized by a population of B cells.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994). Single-chain antibodies are disclosed, for example in WO 88/06630 and WO 92/01047.

The term “diabody” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The term “bispecific antibody” refers to an antibody that shows specificities to two different types of antigens. The term as used herein specifically includes, without limitation, antibodies which show binding specificity for a target antigen and to another target that facilitates delivery to a particular tissue. Similarly, multi-specific antibodies have two or more binding specificities.

The expression “linear antibody” is used to refer to comprising a pair of tandem Fd segments (V_{H}-C_{H}1-V_{H}-C_{H}1) which form a pair of antigen binding regions. Linear antibodies can be bispecific or monospecific and are described, for example, by Zapata et al., Protein Eng. 8(10):1057-1062 (1995).

The term “neutralizing antibody” is used herein in the broadest sense and refers to any antibody that inhibits an influenza virus from replicatively infecting a target cell, regardless of the mechanism by which neutralization is achieved. Thus, for example, neutralization can be achieved by inhibiting the attachment or adhesion of the virus to the cell surface, e.g., by engineering an antibody that binds directly to, or close by, the site responsible for the attachment or adhesion of the virus. Neutralization can also be achieved by an antibody directed to the virion surface, which results in the aggregation of virions. Neutralization can further occur by inhibition of the fusion of viral and cellular membranes following attachment of the virus to the target cell, by inhibition of endocytosis, inhibition of progeny virus from the infected cell, and the like. The neutralizing antibodies of the present invention are not limited by the mechanism by which neutralization is achieved.

The term “antibody repertoire” is used herein in the broadest sense and refers to a collection of antibodies or antibody fragments which can be used to screen for a particular property, such as binding ability, binding specificity, ability of gastrointestinal transport, stability, affinity, and the like. The term specifically includes antibody libraries, including all forms of combinatorial libraries, such as, for example, antibody phage display libraries, including, without limitation, single-chain Fv (scFv) and Fab antibody phage display libraries from any source, including naive, synthetic and semi-synthetic libraries.

A “phage display library” is a protein expression library that expresses a collection of cloned protein sequences as fusions with a phage coat protein. Thus, the phrase “phage display library” refers herein to a collection of phage (e.g., filamentous phage) wherein the phage express an external (typically heterologous) protein. The external protein is free to interact with (bind to) other moieties with which the phage are contacted. Each phage displaying an external protein is a “member” of the phage display library.

An “antibody phage display library” refers to a phage display library that displays antibodies or antibody fragments. The antibody library includes the population of phage or a collection of vectors encoding such a population of phage, or cell(s) harboring such a collection of phage or vectors. The library can be monovalent, displaying on average one single-chain antibody or antibody fragment per phage particle, or multi-valent, displaying, on average, two or more antibodies or antibody fragments per viral particle. The term “antibody fragment” includes, without limitation, single-chain Fv (scFv) fragments and Fab fragments. Preferred antibody libraries comprise on average more than 10⁶, or more than 10⁷, or more than 10⁸, or more than 10⁹ different members.

The term “filamentous phage” refers to a viral particle capable of displaying a heterogenous polypeptide on its surface, and includes, without limitation, f1, fd, Pf1, and M13. The filamentous phage may contain a selectable marker such as tetracycline (e.g., “fd-tet”). Various filamentous phage display systems are well known to those of skill in the art (see, e.g., Zacher et al., Gene 9:127-140 (1980), Smith et al., Science 228:1315-1317 (1985); and Parmley and Smith, Gene 73:305-318 (1988)).

The term “panning” is used to refer to the multiple rounds of screening process in identification and isolation of phages carrying compounds, such as antibodies, with high affinity and specificity to a target.

The term “non-human animal” as used herein includes, but is not limited to, mammals such as, for example, non-human primates, rodents (e.g., mice and rats), and non-rodent animals, such as, for example, rabbits, pigs, sheep, goats, cows, pigs, horses and donkeys. It also includes birds (e.g., chickens, turkeys, ducks, geese and the like). The term “non-primate animal” as used herein refers to mammals other than primates, including but not limited to the mammals specifically listed above.

The phrase “functionally different antibodies,” and grammatical variants thereof, are used to refer to antibodies that differ from each other in at least one property, including, without limitation, binding specificity, binding affinity, and any immunological or biological function, such as, for example, ability to neutralize a target, extent or quality of biological activity, etc.

The phrase “conserved amino acid residues” is used to refer to amino acid residues that are identical between two or more amino acid sequences aligned with each other.

### B. General Techniques

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; Antibody Phage Display: Methods and Protocols, P. M. O'Brian and R. Aitken, eds., Humana Press, In: Methods in Molecular Biology, Vol. 178; Phage Display: A Laboratory Manual, C. F. Barbas III et al. eds., Cold Spring Harbor, N.Y., USA, Cold Spring Harbor Laboratory Press, 2001; and Antibodies, G. Subramanian, ed., Kluwer Academic, 2004. Mutagenesis can, for example, be performed using site-directed mutagenesis (Kunkel et al., Proc. Natl. Acad. Sci USA 82:488-492 (1985)).

In the following description, the invention is illustrated with reference to certain types of antibody libraries, but the invention is not limited to the use of any particular type of antibody library. Recombinant monoclonal antibody libraries can be based on immune fragments or naive fragments. Antibodies from immune antibody libraries are typically constructed with V_{H} and V_{L} gene pools that are cloned from source B cells into an appropriate vector for expression to produce a random combinatorial library, which can subsequently be selected for and/or screened. Other types of libraries may be comprised of antibody fragments from a source of genes that is not explicitly biased for clones that bind to an antigen. Thus, naive antibody libraries derive from natural, unimmunized, rearranged V genes. Synthetic antibody libraries are constructed entirely by in vitro methods, introducing areas of complete or tailored degeneracy into the CDRs of one or more V genes. Semi-synthetic libraries combine natural and synthetic diversity, and are often created to increase natural diversity while maintaining a desired level of functional diversity. Thus, such libraries can, for example, be created by shuffling natural CDR regions (Soderlind et al., Nat. Biotechnol. 18:852-856 (2000)), or by combining naturally rearranged CDR sequences from human B cells with synthetic CDR1 and CDR2 diversity (Hoet et al., Nat. Biotechnol 23:455-38 (2005)). The present invention encompasses the use of naïve, synthetic and semi-synthetic antibody libraries, or any combination thereof.

Similarly, the methods of the present invention are not limited by any particular technology used for the display of antibodies. Although the invention is illustrated with reference to phage display, antibodies of the present invention can also be identified by other display and enrichment technologies, such as, for example, ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), microbial cell display, such as bacterial display (Georgiou et al., Nature Biotech. 15:29-34 (1997)), or yeast cell display (Kieke et al., Protein Eng. 10:1303-1310 (1997)), display on mammalian cells, spore display, viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

In ribosome display, the antibody and the encoding mRNA are linked by the ribosome, which at the end of translating the mRNA is made to stop without releasing the polypeptide. Selection is based on the ternary complex as a whole.

In a mRNA display library, a covalent bond between an antibody and the encoding mRNA is established via puromycin, used as an adaptor molecule (Wilson et al., Proc. Natl. Acad. Sci. USA 98:3750-3755 (2001)). For use of this technique to display antibodies, see, e.g., Lipovsek and Pluckthun, J. Immunol. Methods. 290:51-67 (2004).

Microbial cell display techniques include surface display on a yeast, such as Saccharomyces cerevisiae (Boder and Wittrup, Nat. Biotechnol. 15:553-557 (1997)). Thus, for example, antibodies can be displayed on the surface of S. cerevisiae via fusion to the α-agglutinin yeast adhesion receptor, which is located on the yeast cell wall. This method provides the possibility of selecting repertoires by flow cytometry. By staining the cells by fluorescently labeled antigen and an anti-epitope tag reagent, the yeast cells can be sorted according to the level of antigen binding and antibody expression on the cell surface. Yeast display platforms can also be combined with phage (see, e.g., Van den Beucken et al., FEBS Lett. 546:288-294 (2003)).

For a review of techniques for selecting and screening antibody libraries see, e.g., Hoogenboom, Nature Biotechnol. 23(9):1105-1116 (2005).

### C. Detailed Description of Preferred Embodiments

The present invention concerns the selection, production and use of monoclonal antibodies neutralizing more than one strain (isolate) of an influenza A subtype, including isolates of extinct strains, as well as neutralizing antibodies to more than one influenza A subtype, including subtypes characterized by the presence of an H5 hemagglutinin. In a particular embodiment, the invention concerns the selection, production and use of monoclonal antibodies neutralizing more than one influenza A subtypes and/or more than one isolate, or more than two isolates, or more than three isolates, or more than four isolates, or more than five isolates, etc., most preferably all isolates of one or more subtypes.

The virions of influenza A virus contain 8 segments of linear negative-sense single stranded RNA. The total genome length is 13600 nucleotides, and the eight segments are 2350 nucleotides; 2350 nucleotides; of 2250 nucleotides; 1780 nucleotides; 1575 nucleotides; 1420 nucleotides; 1050 nucleotides; and 900 nucleotides, respectively, in length. Host specificity and attenuation of influenza A virus have been attributed to viral hemagglutinin (H, HA), nucleoprotein (NP), matrix (M), and non-structural (NS) genes individually or in combinations of viral genes (see, e.g., Rogers et al., Virology 127:361-373 (1983); Scholtissek et al., Virology 147:287-294 (1985); Snyder et al., J. Clin. Microbiol. 24:467-469 (1986); Tian et al., J. Virol 53:771-775 (1985); Treanor et al., Virology 171:1-9 (1989).

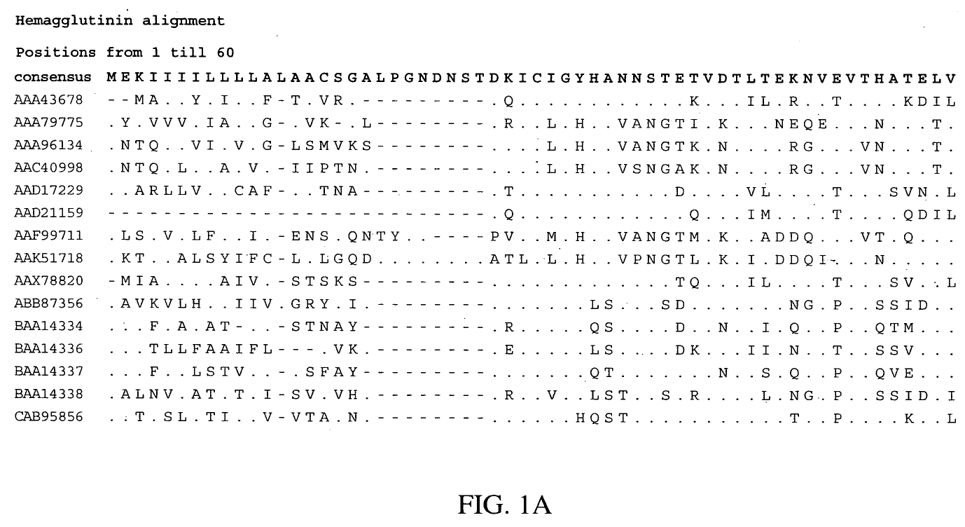

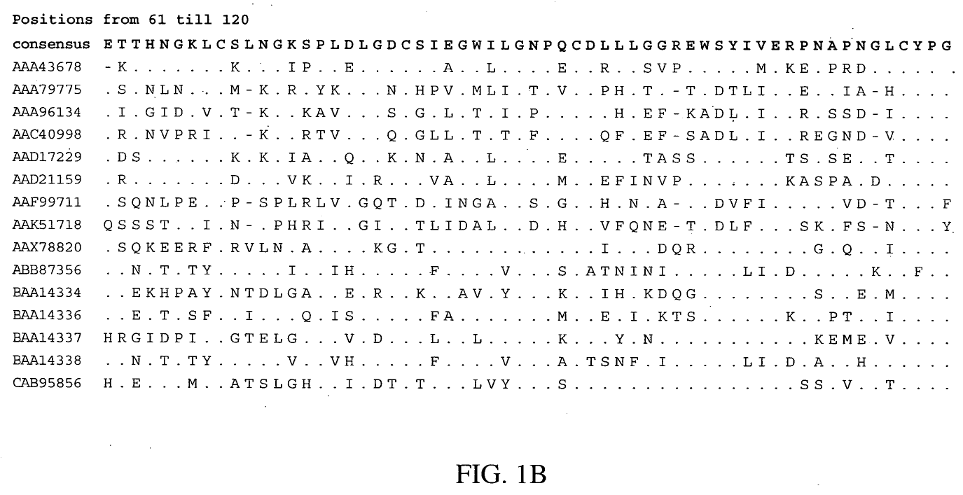

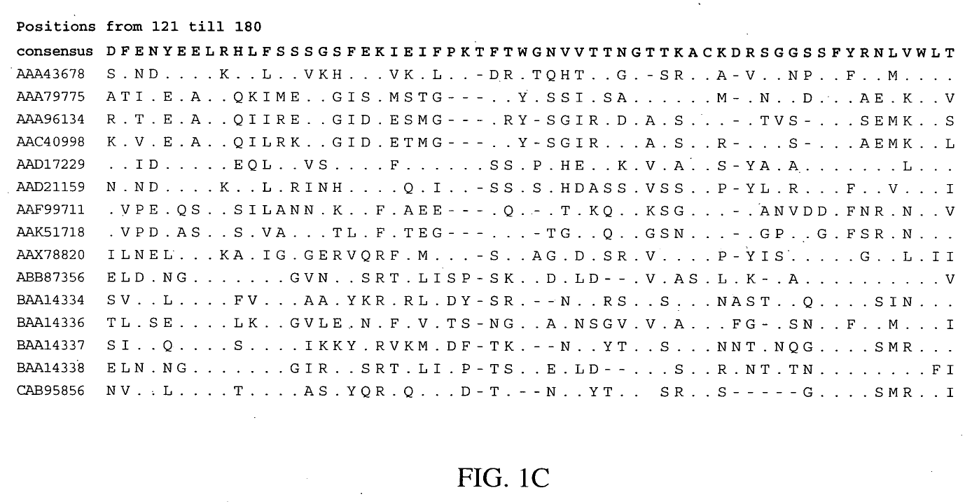

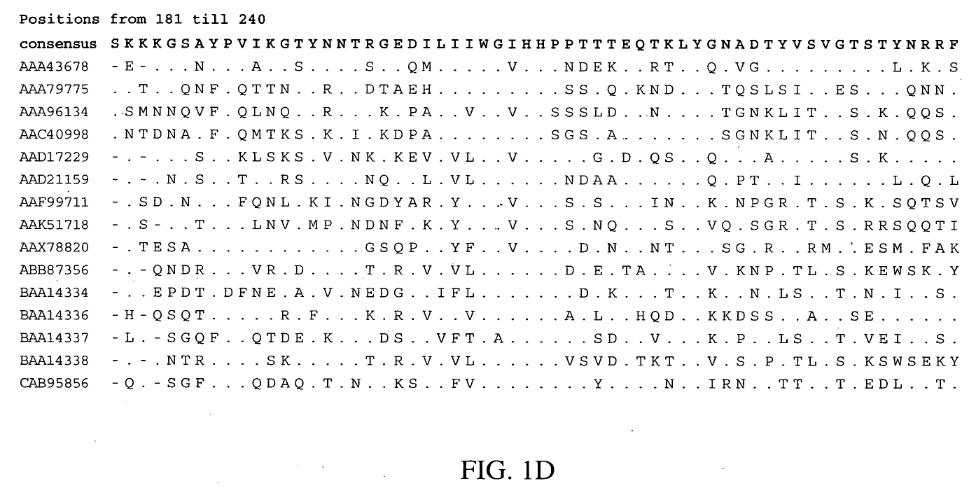

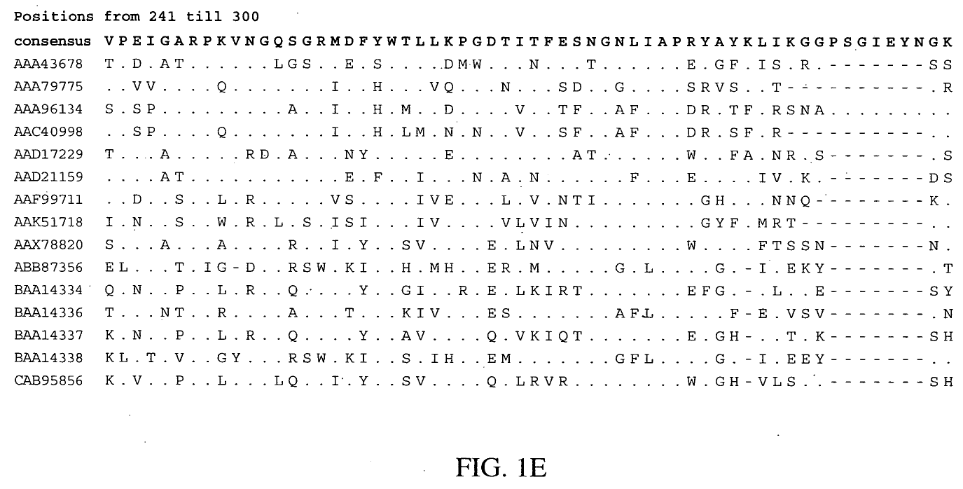

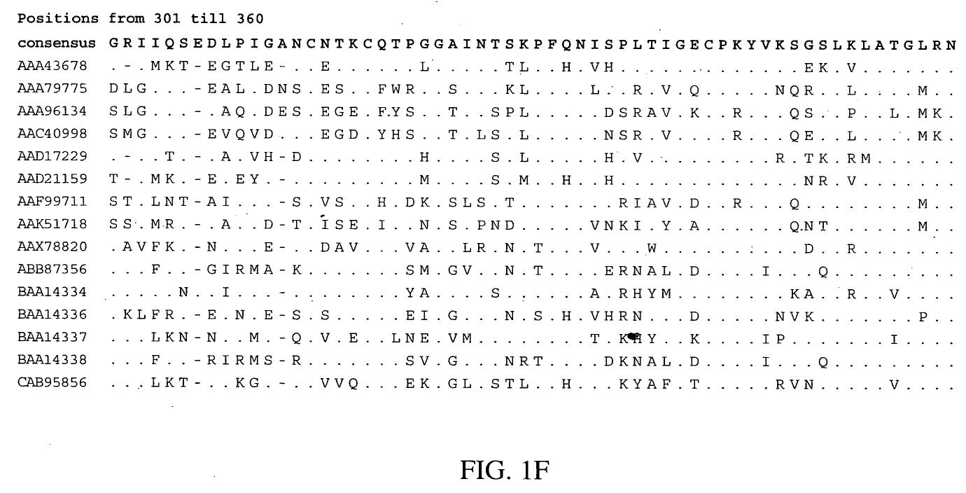

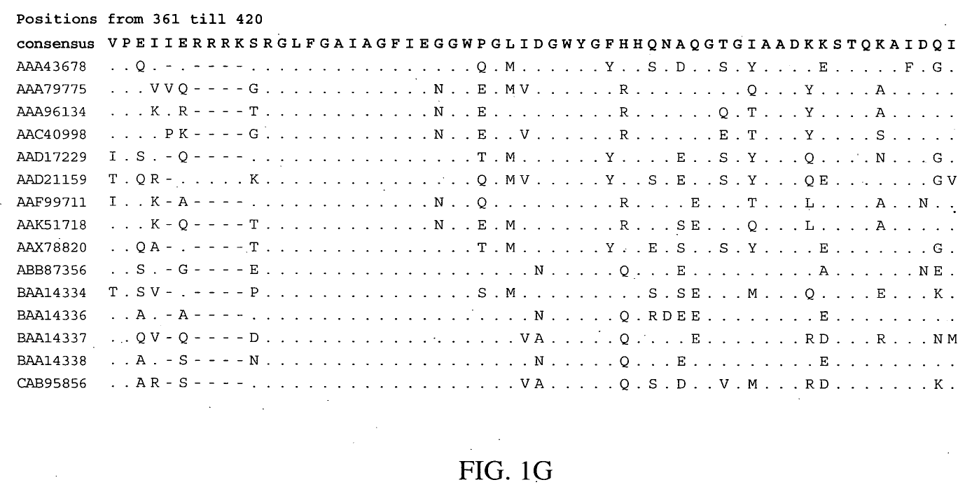

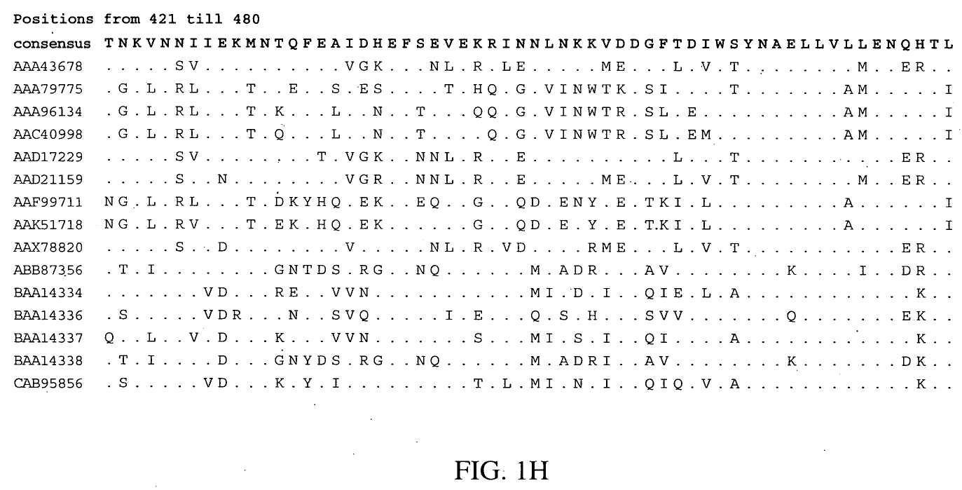

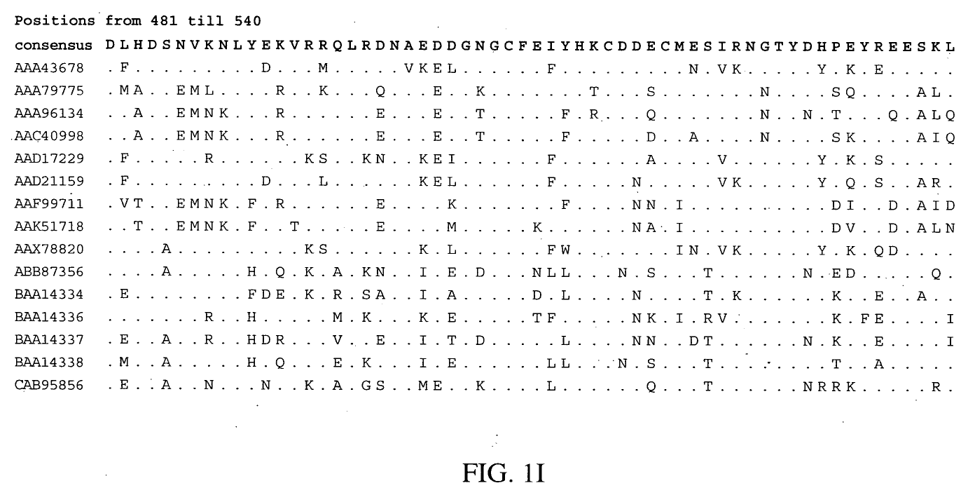

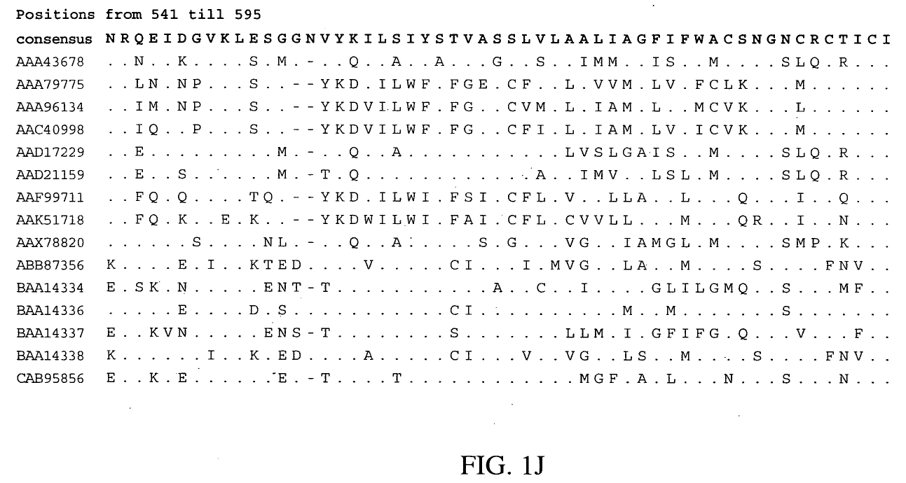

Nucleotide and amino acid sequences of influenza A viruses and their surface proteins, including hemagglutinins and neuraminidase proteins, are available from GenBank and other sequence databases, such as, for example, the Influenza Sequence Database maintained by the Theoretical Biology and Biophysics Group of Los Alamos National Laboratory. The amino acid sequences of 15 known H subtypes of the influenza A virus hemagglutinin (H1-H15) are shown in FIG. 1 (SEQ ID NOS: 1-15). An additional influenza A virus hemagglutinin subtype (H16) was isolated recently from black-headed gulls in Sweden, and reported by Fouchier et al., J. Virol. 79(5):2814-22 (2005). A large variety of strains of each H subtype are also known. For example, the sequence of the HA protein designated H5 A/Hong Kong/156/97 in FIG. 1 was determined from an influenza A H5N1 virus isolated from a human in Hong Kong in May 1997, and is shown in comparison with sequences of several additional strains obtained from other related H5N1 isolates in Suarez et al., J. Virol. 72:6678-6688 (1998).

The structure of the catalytic and antigenic sites of influenza virus neuraminidase have been published by Colman et al., Nature 303:41-4 (1983), and neuraminidase sequences are available from GenBank and other sequence databases.

It has been known that virus-specific antibodies resulting from the immune response of infected individuals typically neutralize the virus via interaction with the viral hemagglutinin (Ada et al., Curr. Top. Microbiol. Immunol. 128:1-54 (1986); Couch et al., Annu. Rev. Micobiol. 37:529-549 (1983)). The three-dimensional structures of influenza virus hemagglutinins and crystal structures of complexes between influenza virus hemagglutinins and neutralizing antibodies have also been determined and published, see, e.g., Wilson et al., Nature 289:366-73 (1981); Ruigrok et al., J. Gen. Virol. 69 (Pt 11):2785-95 (1988); Wrigley et al., Virology 131(2):308-14 (1983); Daniels et al., EMBO J. 6:1459-1465 (1987); and Bizebard et al., Nature 376:92-94 (2002).

According to the present invention, antibodies with the desired properties are identified from one or more antibody libraries, which can come from a variety of sources and can be of different types.

Comprehensive Human Influenza Antibody Libraries

Comprehensive human influenza antibody libraries can be created from antibodies obtained from convalescent patients of various prior influenza, seasonal outbreaks epidemics, and pandemics, including the 1968 Hong Kong flu (H3N2), the 1957 Asian flu (H2N2), the 1918 Spanish flu (H1N1), and the 2004/2005 Avian flu (H5N1). In order to prepare such libraries, blood or bone marrow samples are collected from individuals known or suspected to have been infected with an influenza virus. Peripheral blood samples, especially from geographically distant sources, may need to be stabilized prior to transportation and use. Kits for this purpose are well known and commercially available, such as, for example, BD Vacutainer® CPT™ cell preparation tubes can be used for centrifugal purification of lymphocytes, and guanidium, Trizol, or RNAlater used to stabilize the samples. Upon receipt of the stabilized lymphocytes or whole bone marrow, RT-PCR is performed to rescue heavy and light chain repertoires, using immunoglobulin oligo primers known in the art. The PCR repertoire products are combined with linker oligos to generate scFv libraries to clone directly in frame with m13 pIII protein, following procedures known in the art.

In a typical protocol, antibodies in the human sera can be detected by well known serological assays, including, for example, by the well-known hemagglutinin inhibition (HAI) assay (Kendal, A. P., M. S. Pereira, and J. J. Skehel. 1982. Concepts and procedures for laboratory-based influenza surveillance. U.S. Department of Health and Human Services, Public Health Service, Centers for Disease Control, Atlanta, Ga.), or the microneutralization assay (Harmon et al., J. Clin. Microbiol. 26:333-337 (1988)). This detection step might not be necessary if the serum sample has already been confirmed to contain influenza neutralizing antibodies. Lymphocytes from whole blood or those present in bone marrow are next processed by methods known in the art. Whole RNA is extracted by Tri BD reagent (Sigma) from fresh or RNAlater stabilized tissue. Subsequently, the isolated donor total RNA is further purified to mRNA using Oligotex purification (Qiagen). Next first strand cDNA synthesis, is generated by using random nonamer oligonucleotides and or oligo (dT)₁₈ primers according to the protocol of AccuScript reverse transcriptase (Stratagene). Briefly, 100 ng mRNA, 0.5 mM dNTPs and 300 ng random nonamers and or 500 ng oligo (dT)₁₈ primers in Accuscript RT buffer (Stratagene) are incubated at 65° C. for 5 min, followed by rapid cooling to 4° C. Then, 100 mM DTT, Accuscript RT, and RNAse Block are added to each reaction and incubated at 42° C. for 1 h, and the reverse transcriptase is inactivated by heating at 70° C. for 15 minutes. The cDNA obtained can be used as a template for RT-PCR amplification of the antibody heavy and light chain V genes, which can then be cloned into a vector, or, if phage display library is intended, into a phagemid vector. This procedure generates a repertoire of antibody heavy and light chain variable region clones (V_{H} and V_{L} libraries), which can be kept separate or combined for screening purposes.

Immunoglobulin repertoires from peripheral lymphocytes of survivors of earlier epidemics and pandemics, such as the 1918 Spanish Flu, can be retrieved, stabilized, and rescued in a manner similar to that described above. For additional H1 and H3 libraries repertoires can be recovered from properly timed vaccinated locally-sourced donors. As an additional option commercially available bone marrow total RNA or mRNA can be purchased from commercial sources to produce libraries suitable for H1 and H3, and depending upon the background of donor also suitable for H2 antibody screening.

Universal Antibody Library (UAL)—Synthetic Human-Like Repertoire

In the methods of the present invention, the synthetic human antibody repertoire can be represented by a universal antibody library, which can be made by methods known in the art or obtained from commercial sources. Thus, for example, universal immunoglobulin libraries, including subsets of such libraries, are described in U.S. Patent Application Publication No. 20030228302 published on Dec. 11, 2003, the entire disclosure of which is hereby expressly incorporated by reference. In brief, this patent publication describes libraries of a prototype immunoglobulin of interest, in which a single predetermined amino acid has been substituted in one or more positions in one or more complementarity-determining regions of the immunoglobulin of interest. Subsets of such libraries include mutated immunoglobulins in which the predetermined amino acid has been substituted in one or more positions in one or more of the six complementarity-determining regions of the immunoglobulin in all possible combinations. Such mutations can be generated, for example, by walk-through mutagenesis, as described in U.S. Pat. Nos. 5,798,208, 5,830,650, 6,649, 340, and in U.S. Patent Application Publication No. 20030194807, the entire disclosures of which are hereby expressly incorporated by reference. In walk-through mutagenesis, a library of immunoglobulins is generated in which a single predetermined amino acid is incorporated at least once into each position of a defined region, or several defined regions, of interest in the immunoglobulin, such as into one or more complementarity determining regions (CDRs) or framework (FR) regions of the immunoglobulins. The resultant mutated immunoglobulins differ from the prototype immunoglobulin, in that they have the single predetermined amino acid incorporated into one or more positions within one or more regions (e.g., CDRs or FR region) of the immunoglobulin, in lieu of the “native” or “wild-type” amino acid which was present at the same position or positions in the prototype immunoglobulin. The set of mutated immunoglobulins includes individual mutated immunoglobulins for each position of the defined region of interest; thus, for each position in the defined region of interest (e.g., the CDR or FR) each mutated immunoglobulin has either an amino acid found in the prototype immunoglobulin, or the predetermined amino acid, and the mixture of all mutated immunoglobulins contains all possible variants.

Specific sublibraries of antibody heavy and light chains with various mutations can be combined to provide the framework constructs for the antibodies of the present invention, which is followed by introducing diversity in the CDRs of both heavy and light chains. This diversity can be achieved by methods known in the art, such as, for example, by Kunkel mutagenesis, and can be repeated several times in order to further increase diversity. Thus, for example, diversity into the heavy and light chain CDR1 and CD2 regions, separately or simultaneously, can be introduced by multiple rounds of Kunkel mutagenesis. If necessary, the various Kunkel clones can be segregated by CDR lengths and/or clones lacking diversity in a targeted CDR (e.g., CDR1 or CDR3) can be removed, e.g., by digestion with template-specific restriction enzymes. Upon completion of these steps, the size of the library should exceed about 10⁹ members, but libraries with lesser members are also useful.

In a specific embodiment, both immunized antibody libraries and universal antibody libraries are used for identifying the neutralizing antibodies of the present invention. The two types of libraries are fundamentally different. The universal antibody libraries are retrospectively synthesized collections of human-like antibodies with the predicted ability to bind proteins and peptides, while an immunized repertoire will contain sequences to specifically recognize avian H5 hemagglutinin, and/or H1, H2, or H3 hemagglutinin, as the case may be. Thus, the immunized repertoires are theoretically optimized to recognize critical components of targeted influenza subtype(s). As a result these differences the two methods produce a different set of antibodies and thus provide a more efficient approach for identifying the desired neutralizing antibodies.

Hyperimmunized Non-Human Primate Antibody Libraries

In this method, an antibody library is rescued from hyperimmunized non-human primates, such as, for example, macaque or baboons. Specifically, non-human primates are immunized with various subtypes of the influenza A virus or with various hemagglutinin (H) proteins. Animals developing titers of antibody recognizing the influenza A virus subtype or hemagglutinin they were immunized with are sacrificed and their spleens harvested. Blood or bone marrow of the immunized animals is collected, and antibodies produced are collected and amplified as described above for the comprehensive influenza antibody libraries.

Strategies for Isolating Neutralizing Antibodies of the Invention

Regardless of the type of antibody library or libraries used, antibodies with dual specificities, such as, for example, showing reactivity with two different influenza A subtypes and/or with two strains (isolates) of the same subtype, and/or with human and non-human isolates, can be discovered and optimized through controlled cross-reactive selection and/or directed combinatorial and/or mutagenic engineering.

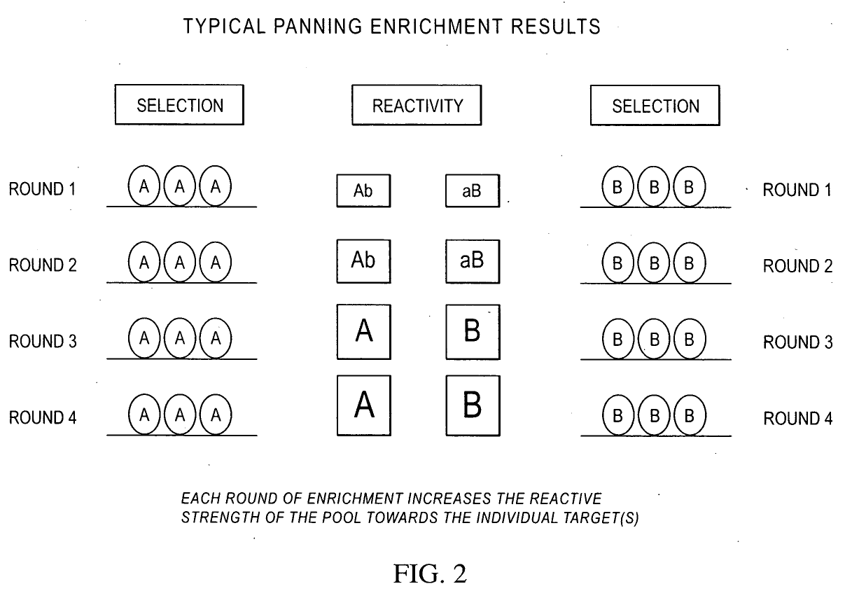

In a typical enrichment scheme, illustrated in FIG. 2, a library including antibodies showing cross-reactivity to two targets, designated as targets A and B, are subjected to multiple rounds of enrichment. If enrichment is based on reactivity with target A, each round of enrichment will increase the reactive strength of the pool towards target A. Similarly, if enrichment is based on reactivity with target B, each round of enrichment will increase the reactive strength of the pool towards target B. Although FIG. 2 refers to panning, which is the selection method used when screening phage display libraries (see below), the approach is equally applicable to any type of library discussed above, other otherwise known in the art, and to any type of display technique. Targets A and B include any targets to which antibodies bind, including but not limited to various isolates, types and sub-types of influenza viruses.

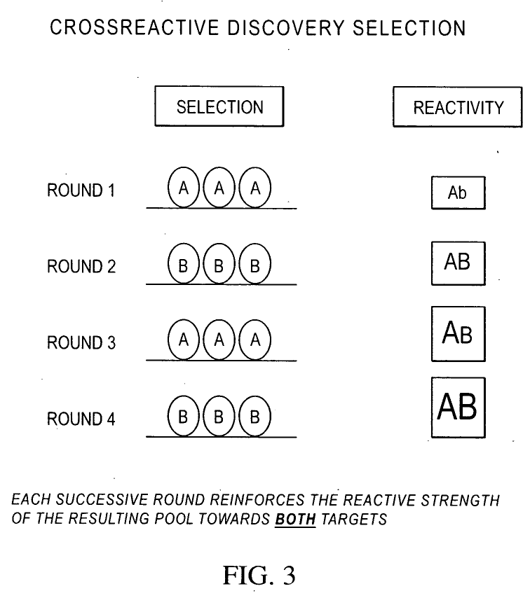

Since the goal of the present invention is to identify neutralizing antibodies with multiple specificities, a cross-reactive discovery selection scheme has been developed. In the interest of simplicity, this scheme is illustrated in FIG. 3 showing the selection of antibodies with dual specificities. In this case, an antibody library including antibodies showing reactivity with two targets, targets A and B, is first selected for reactivity with one of the targets, e.g., target A, followed by selection for reactivity with the other target, e.g., target B. Each successive selection round reinforces the reactive strength of the resulting pool towards both targets. Accordingly, this method is particularly useful for identifying antibodies with dual specificity. Of course, the method can be extended to identifying antibodies showing reactivity towards further targets, by including additional rounds of enrichment towards the additional target(s). Again, if the library screened is a phage display library, selection is performed by cross-reactive panning, but other libraries and other selection methods can also be used.

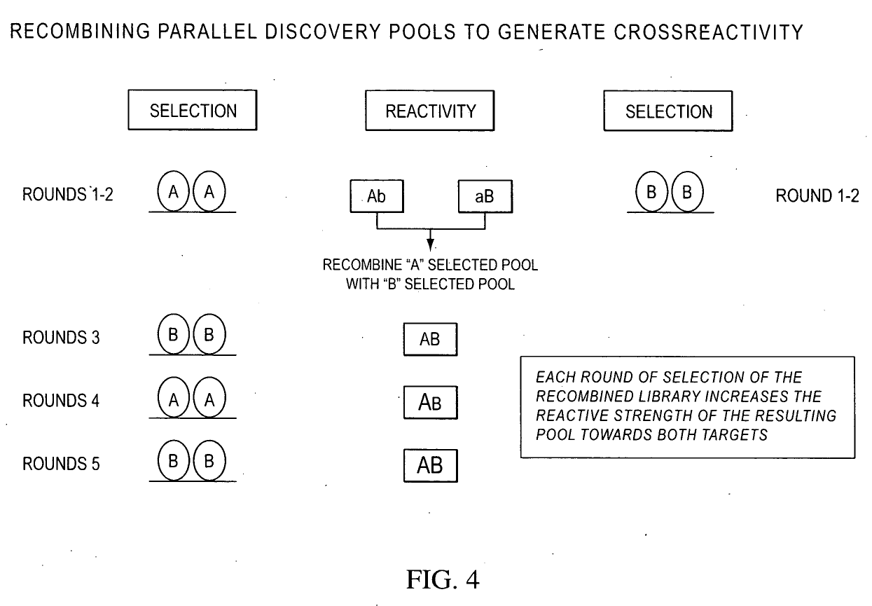

A combination of the two methods discussed above includes two separate enrichment rounds for reactivity towards target A and target B, respectively, recombining the two pools obtained, and subsequent cross-reactive selection rounds, as described above. This approach is illustrated in FIG. 4. Just as in the pure cross-reactive selection, each round of selection of the recombined library increases the reactive strength of the resulting pool towards both targets.

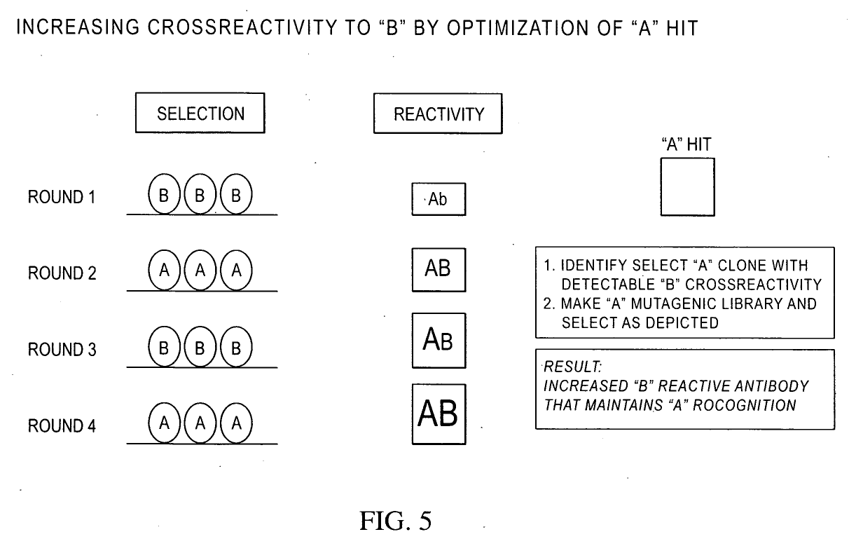

In a further embodiment, illustrated in FIG. 5, first a clone showing strong reactivity with a target A, and having detectable cross-reactivity with target B is identified. Based on this clone, a mutagenic library is prepared, which is then selected, in alternating rounds, for reactivity with target B and target A respectively. This scheme will result in antibodies that maintain strong reactivity with target A, and have increased reactivity with target B. Just as before, selection is performed by panning, if the libraries screened are phage display libraries, but other libraries, other display techniques, and other selection methods can also be used, following the same strategy.

As discussed above, targets A and B can, for example, be two different subtypes of the influenza A virus, two different strains (isolates) of the same influenza A virus, subtypes or isolates from two different species, where one species is preferably human. Thus, for example, target A may be an isolate of the 2004 Vietnam isolate of the H5N1 virus, and target B may be a 1997 Hong Kong isolate of the H5N1 virus. It is emphasized that these examples are merely illustrative, and antibodies with dual and multiple specificities to any two or multiple targets can be identified, selected and optimized in an analogous manner.

Alternatively, if an antibody library such as the UAL that allows segregation of discrete frameworks and CDR lengths is used to find an antibody to target A, then an antigen B could be screened for and the library could be restricted to a diverse collection of similar parameters. Once an antibody to antigen B is found then chimeric or mutagenic antibodies based upon the respective A and B antibodies could be used to engineer a dual specific collection.

Phage Display

In a particular embodiment, the present invention utilizes phage display antibody libraries to functionally discover neutralizing monoclonal antibodies with multiple (including dual) specificities. Such antibodies can, for example, be monoclonal antibodies capable of neutralizing more than one influenza A virus subtype, including the H5, H7 and/or H9 subtypes, such as the H5 and H1; H5 and H2; H5 and H3; H5, H1, and H2; H5, H1, and H3; H5, H2 and H3; H1, H2 and H3, etc., subtypes, and/or more than one strain (isolate) of the same subtype.

To generate a phage antibody library, a cDNA library obtained from any source, including the libraries discussed above, is cloned into a phagemid vector.

Thus, for example, the collection of antibody heavy and light chain repertoires rescued from lymphocytes or bone marrow by RT-PCR as described above, is reassembled as a scFv library fused to m13 pIII protein. The combinatorial library will contain about more than 10⁶, or more than 10⁷, or more than 10⁸, or more than 10⁹ different members, more than 10⁷ different members or above being preferred. For quality control random clones are sequenced to assess overall repertoire complexity.

Similarly, following the initial PCR rescue of heavy and light chain variable regions from a naive or immunized human, or hyperimmunized nonhuman primate antibody library, the PCR products are combined with linker oligos to generate scFv libraries to clone directly in frame with M13 pIII coat protein. The library will contain about more than 10⁶, or more than 10⁷, or more than 10⁸, or more than 10⁹ different members, more than 10⁷ different members or above being preferred. As a quality control step, random clones are sequenced in order to assess overall repertoire size and complexity.

Antibody phage display libraries may contain antibodies in various formats, such as in a single-chain Fv (scFv) or Fab format. For review see, e.g., Hoogenboom, Methods Mol. Biol. 178:1-37 (2002).

Screening

Screening methods for identifying antibodies with the desired neutralizing properties have been described above. Reactivity can be assessed based on direct binding to the desired hemagglutinin proteins.

Hemagglutinin (HA) Protein Production

Hemagglutinin (HA) proteins can be produced by recombinant DNA technology. In this method, HA genes are cloned into an appropriate vector, preferably a baculovirus expression vector for expression in baculovirus-infected insect cells, such as Spodoptera frugiperda (Sf9) cells.

The nucleic acid coding for the HA protein is inserted into a baculovirus expression vector, such as Bac-to-Bac (Invitrogen), with or without a C-terminal epitope tag, such as a poly-his (hexahistidine tag). A poly-his tag provides for easy purification by nickel chelate chromatography.

In general the cloning involves making reference cDNAs by assembly PCR from individually synthesized oligos. Corresponding isolate variant HA proteins are made by either substituting appropriate mutant oligos into additional assembly PCRs or by mutagenesis techniques, such as by Kunkel mutagenesis. Two clusters of HA protein sequences exist for H5, the 1997 and 2004 subtype isolates. Therefore, a single reference protein is made for each cluster. Similarly, reference proteins are generated for 1918 Spanish flu (H1), 1958 Asian Flu (H2), 1968 Hong Kong Flu (H3), and current H1, H2, H3 isolates.

Recombinant baculovirus is generated by transfecting the above Bacmid into Sf9 cells (ATCC CRL 1711) using lipofectin (commercially available from Gibco-BRL). After 4-5 days of incubation at 28° C., the released viruses are harvested and used for further amplifications. Viral infection and protein expression are performed as described by O'Reilley et al., Baculovirus Expression Vectors: A Laboratory Manual (Oxford: Oxford University Press, 1994).

Expressed poly-His-tagged HA polypeptides can then be purified, for example, by Ni²⁺-chelate affinity chromatography as follows. Supernantents are collected from recombinant virus-infected Sf9 cells as described by Rupert et al., Nature 362:175-179 (1993). A Ni²⁺-NTA agarose column (commercially available from Qiagen) is prepared with a bed volume of 5 mL, washed with 25 mL of water, and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes non-specifically bound protein. After reaching A₂₈₀ baseline again, the column is developed with a 0 to 500 mM imidazole gradient in the secondary wash buffer. One-mL fractions are collected and analyzed by SDS-PAGE and silver staining or Western blot with Ni²⁺-NTA-conjugated to alkaline phosphatase (Qiagen). Fractions containing the eluted His₁₀-tagged HA polypeptide are pooled and dialyzed against loading buffer.

Alternatively, purification of an IgG-tagged (or Fc-tagged) HA polypeptide can be performed using known chromatography techniques, including, for instance, Protein A or protein G column chromatography.

As an alternative to using Sf9 cells HA proteins can also be produced in other recombinant host cells, prokaryote, yeast, or higher eukaryote cells. Suitable prokaryotes include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Various E. coli strains are publicly available, such as E. Coli K12 strain MM294 (ATCC 31,446); E. coli X1776 (ATCC 31,537); E. coli strain W3110 (ATCC 27,325); and K5 772 (ATCC 53,635).

In addition to prokaryotes, eukaryotic microbes such as filamentous fungi or yeast are suitable cloning or expression hosts for vectors containing nucleic acid encoding an HA polypeptide. Saccharomyces cerevisiae is a commonly used lower eukaryotic host microorganism. However, a number of other genera, species, and strains are commonly available and useful herein, such as Schizosaccharomyces pombe (Beach and Nurse, Nature 290: 140 (1981); EP 139,383 published 2 May 1985); Kluyveromyces hosts (U.S. Pat. No. 4,943,529; Fleer et al., Bio/Technology 9:968-975 (1991)) such as, e.g., K. lactis (MW98-8C, CBS683, CBS4574; Louvencourt et al., J. Bacteriol. 737 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology 8:135 (1990)), K. thermotolerans, and K. marxianus; yarrowia (EP 402,226); Pichia pastoris (EP 183,070; Sreekrishna et al., J. Basic Microbiol. 28:265-278 (1988)); Candida; Trichoderma reesia (EP 244,234); Neurospora crassa (Case et al., Proc. Natl. Acad. Sci. USA 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun. 112:284-289 (1983); Tilburn et al., Gene 26:205-221 (1983); Yelton et al., Proc. Nat. Acad. Sci. USA 81:1470-1474 (1984)) and A. niger Kelly and Hynes, EMBO J. 4:475-479 (1985). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs 269 (1982).

Suitable host cells for the expression of HA proteins include cells of multicellular organisms. Examples of invertebrate cells include the above-mentioned insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (HEK 293 or HEK 293 cells subcloned for growth in suspension culture (Graham et al., J. Gen Virol. 36:59 (1977)); Chinese hamster ovary cells/-DHFR (CHO, Urlaub and Chasin, Proc. Natl. Acad. Sci. USA 77:4216 (1980)); mouse sertoli cells (TM4, Mather, Biol. Reprod. 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

Hemagglutinin (HA) Protein Panning

HA protein is immobilized on to the surface of microtiter wells or magnetic beads to pan the described above libraries. In a particular embodiment, each library is allowed to bind the H5 protein at 4 degrees for two hours and then washed extensively with cold PBS, before eluting HA specific binding clones with 0.2M glycine-HCl buffer (pH2.5). The recovered phage is pH neutralized and amplified by infecting a susceptible host E. coli. Subsequently, phagemid production can be induced to repeat the enrichment of positive clones and subsequent clones isolation for triage. Upon sufficient enrichment the entire pool is transferred by infection into a non amber suppressor E. Coli strain such as HB2151 to express soluble scFv proteins. Alternatively the pool(s) could be subcloned into a monomeric scFv expression vector, such as pBAD, and recombinant soluble scFv proteins are expressed for in vitro analysis and characterization, as described below.

Characterization

H5 clones are first tested for binding affinity to an H5 protein produced as described above. In a particular example, binding is tested to a 2004 H5 protein (Refseq AAS65618, Isolate; A/Thailand/2(SP-33)/2004(H5N1)), and in parallel test to a 1997 H5 protein (Refseq AAF74331, Isolate; A/Hong Kong/486/97(H5N1)), but other isolates can also be used alone or in any combination. The positive clones obtained with the 2004 and the 1997 H5 proteins will fall into two broad categories: 2004 selective and 2004/1997 nonselective. The typical functional test for neutralization involves hemagglutination inhibition assays using whole virus binding to red blood cells. Due to safety concerns, alternative hemagglutination assays with recombinant protein and red blood cells are preferred. In order to eliminate the need for whole blood, the hemagglutinin binding inhibition assay can be preformed on airway epithelial cells. The binding assay can be performed in any configuration, including, without limitation, any flow cytometric or cell ELISA (cELISA) based assays. Using cELISA is advantageous in that it obviates the use of expensive flow cytometry equipment and can provide for more automated clonal assessment and greater data collection. On the other hand, flow cytometry may provide greater sensitivity, consistency, and speed.

H1 clones can be tested for binding to any H1 proteins, including binding to the current 2004 H1 and, in parallel, for binding to 1918 and 1976 proteins. The positive clones will fall into two broad categories: 2004 selective and 2004 nonselective. Once again it is critical to test for neutralization, using methodologies similar to those described above.

Other HA proteins, such as H2 and H3, can be characterized in an analogous manner.

Optimization

For the efficient management of influenza epidemics and pandemics, including a potential pandemic associated with human infections caused by an avian (H5) virus, antibodies that effectively neutralize current isolates of the H proteins, such as the H5 protein, as well as future mutations, are needed. In order to achieve this goal, diverse H (e.g., H5) neutralizing clones need to be identified that bind all known isolates of the targeted hemagglutinin subtype(s).

If desired, cross-reactivity can be further improved by methods known in the art, such as, for example, by Look Through Mutagenesis (LTM), as described in US. Patent Application Publication No. 20050136428, published Jun. 23, 2005, the entire disclosure of which is hereby expressly incorporated by reference.

Look-through mutagenesis (LTM) is a multidimensional mutagenesis method that simultaneously assesses and optimizes combinatorial mutations of selected amino acids. The process focuses on a precise distribution within one or more complementarity determining region (CDR) domains and explores the synergistic contribution of amino acid side-chain chemistry. LTM generates a positional series of single mutations within a CDR where each wild type residue is systematically substituted by one of a number of selected amino acids. Mutated CDRs are combined to generate combinatorial single-chain variable fragment (scFv) libraries of increasing complexity and size without becoming prohibitive to the quantitative display of all variants. After positive selection, clones with improved properties are sequenced, and those beneficial mutations are mapped. To identify synergistic mutations for improved HA binding properties, combinatorial libraries (combinatorial beneficial mutations, CBMs) expressing all beneficial permutations can be produced by mixed DNA probes, positively selected, and analyzed to identify a panel of optimized scFv candidates. The procedure can be performed in a similar manner with Fv and other antibody libraries.

Mutagenesis can also be performed by walk-through mutagenesis (WTM), as described above.

Another useful mutagenic method to intentionally design cross-reactivity of the antibodies herein with more than one influenza A subtype and/or more than one isolate of the same subtype, is referred herein as “destinational” mutagenesis. Destinational mutagenesis can be used to rationally engineer a collection of antibodies based upon one or more antibody clones, preferably of differing reactivities. In the context of the present invention, destinational mutagenesis is used to encode single or multiple residues defined by analogous positions on like sequences such as those in the individual CDRs of antibodies. In this case, these collections are generated using oligo degeneracy to capture the range of residues found in the comparable positions. It is expected that within this collection a continuum of specificities will exist between or even beyond those of the parental clones. The objective of destinational mutagenesis is to generate diverse multifunctional antibody collections, or libraries, between two or more discrete entities or collections. In the case of influenza this method can be utilized to use two antibodies that recognize two distinct epitopes, isolates, or subtypes and morph both functional qualities into a single antibody. As an example, a first influenza A antibody can be specific to a Vietnam isolate of the H5 subtype and a second antibody is specific to a Thailand or Turkish isolate of the H5 subtype of the influenza A virus. To create a destinational mutagenesis library, the CDR sequences for both antibodies are first attained and aligned. Next all positions of conserved identity are fixed with a single codon to the matched residue. At non-conserved positions a degenerate codon is incorporated to encode both residues. In some instances the degenerate codon will only encode the two parental residues at this position. However, in some instances additional co-products are produced. The level of co-product production can be dialed in to force co-product production or eliminate this production dependent upon size limits or goals.

Thus, for example, if the first position of the two antibodies respectively are threonine and alanine, the degenerate codon with A/G-C- in the first two positions would only encode threonine or alanine, irrespective of the base in the third position. If, for example, the next position residues are lysine and arginine the degenerate codon A-A/G-A/G will only encode lysine or arginine. However, if the degenerate codon A/C-A/G-A/G/C/T were used then asparagine, histidine, glutamine, and serine coproducts will be generated as well.

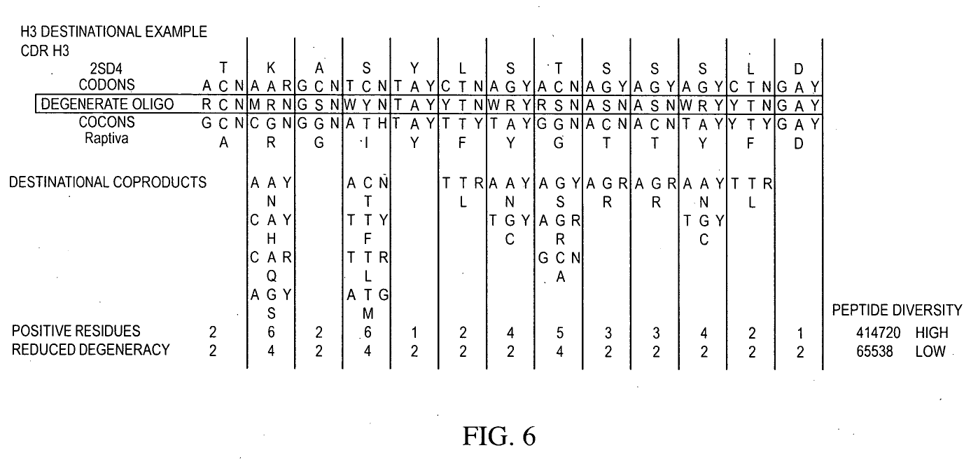

As a convenience it is simpler to use only antibodies with matched CDR lengths. One way to force this is to screen a size restricted library for the second antigen, based on the CDR length and potentially even framework restrictions imparted by the initially discovered antibody. It is noted, however, that using CDRs of equal length is only a convenience and not a requirement. It is easy to see that, while this method will be useful to create large functionally diverse libraries of influenza A virus neutralizing antibodies, its applicability is much broader. This mutagenesis technique can be used to produce functionally diverse libraries or collections of any antibody. Thus, FIG. 6 is included herein to illustrate the use of the destinational mutagenesis method using CDRs of a TNF-α antibody and a CD11a antibody as the parental sequences mutagenized.

Other exemplary mutagenesis methods include saturation mutagenesis and error prone PCR.

Saturation mutagenesis (Hayashi et al., Biotechniques 17:310-315 (1994)) is a technique in which all 20 amino acids are substituted in a particular position in a protein and clones corresponding to each variant are assayed for a particular phenotype. (See, also U.S. Pat. Nos. 6,171,820; 6,358,709 and 6,361,974.)

Error prone PCR (Leung et al., Technique 1:11-15 (1989); Cadwell and Joyce, PCR Method Applic. 2:28-33 (1992)) is a modified polymerase chain reaction (PCR) technique introducing random point mutations into cloned genes. The resulting PCR products can be cloned to produce random mutant libraries or transcribed directly if a T7 promoter is incorporated within the appropriate PCR primer.

Other mutagenesis techniques are also well known and described, for example, in In Vitro Mutagenesis Protocols, J. Braman, Ed., Humana Press, 2001.

In the present case, one of the main goals is to engineer an antibody (or antibodies) to effectively treat current H5 (or H7 or H9) isolates as well as future mutations. To engineer an antibody with tolerances capable of recognizing mutations in new isolates H5 neutralizing clones that bind a variety of H5 isolates, including, for example, both recent 2004 isolates and previous 1997 isolates are to be identified. It is expected that if a clone is selected on a 2004 isolate it will bind/neutralize a 1997 isolate to a lesser degree. In this case the goal is to improve 1997 recognition dramatically within the context of improving (or at least maintaining) 2004 isolate binding. Therefore, selection is first done for improvements on 1997 reference protein followed by selection on the 2004 protein. Doing so provides a greater selective pressure on the new strain, while maintaining pressure on the second parameter.

Optimization can be based on any of the libraries discussed above, or any other types of libraries known in the art, alone or in any combination. In a particular embodiment, optimization can begin by screening three types of LTM libraries; triple mutagenized light chain library, triple mutagenized heavy chain library, and hextuple mutagenized (light+heavy chain) library. H5 is panned essentially as described above, although minor modifications might be desirable. For example, prior to glycine-HCl elution one can select for improved binding by increasing washing stringencies at each round by either or both of the following methods: extensive washing at RT or 37 degrees, or prolonged incubation in presence of excess soluble parent scFv. These selection modifications should improve off-rate kinetics in the resulting clones. After 3-4 rounds of selection we will sequence random clones and test for binding by ELISA. Following sequence analysis of the improved clones, all the allowable improved mutations are combined into a combinatorial beneficial mutagenesis (CBM) library to select for synergistic improvements to binding of both subtype H5 isolates. The CBM library is made by synthesizing degenerate oligo nucleotides to represent all improved and original parental residues at all positions. The resulting library is selected under increasing stringencies, similarly to LTM screening. Following sufficient selection the pool is subcloned into a pBAD expression vector to express and purify monomeric scFv protein from E. coli for binding and neutralization assays, described above.

H1 neutralizing antibodies can be optimized in an analogous manner. In this case one can select and optimize using any reference protein sequences from 1918, 1976, and current as either a starting point or destination.

In addition, intertype recognition is tested with the neutralizing antibody clones. An example of intertype recognition is coincidental or engineered H1 binding from an H5 sourced or optimized clone.

Once neutralizing antibodies with the desired properties have been identified, it might be desirable to identify the dominant epitope or epitopes recognized by the majority of such antibodies. Methods for epitope mapping are well known in the art and are disclosed, for example, in Morris, Glenn E., Epitope Mapping Protocols, Totowa, N.J. ed., Humana Press, 1996; and Epitope Mapping: A Practical Approach, Westwood and Hay, eds., Oxford University Press, 2001.

The handling of antibody libraries, such as libraries from various donors or characterized by reactivity to different isolates of subtypes of a virus, including but not limited to influenza viruses, can be greatly facilitated by applying unique barcodes distinguishing the various antibody collections. The barcodes preferably are selected such that they are capable of propagating along with the clone(s) labeled.

Thus the barcodes can be non-coding DNA sequences of about 1-24 non-coding nucleotides in length that can be deconvoluted by sequencing or specific PCR primers. This way, a collection of nucleic acids, such as an antibody repertoire, can be linked at the cloning step.

In another example, the barcodes are coding sequences of silent mutations. If the libraries utilize restrition enzymes that recognize interrupted palidromes (e.g. Sfi GGCCNNNNNGGCC), distinct nucleotides can be incorporated in place of the “N's” to distinguish various collections of clones, such as antibody libraries. This barcoding approach has the advantage that the repertoire is linked at the amplification step.

In a different example, the barcodes are coding sequences that encode immunologically distinct peptide or protein sequences fused to phage particles. Examples include, for example, epitope (e.g. Myc, HA, FLAG) fusions to pIII, pVIII, pVII, or pIx phages. The epitopes can be used singly or in various combinations, and can be provided in cis (on the library-encoding plasmid) or in trans (specifically modified helper phage) configuration.

Other examples of possible barcodes include, without limitation, chemical and enzymatic phage modifications (for phage libraries) with haptens or fluorescent chromophores. Such tags are preferred for a single round of selection.

While barcoding is illustrated herein for distinguishing antibody libraries, one of ordinary skill will appreciate that the described approaches are broadly applicable for uniquely labeling and distinguishing nucleic acid molecules and collections of nucleic acids in general.

Production of Neutralizing Antibodies

Once antibodies with the desired neutralizing properties are identified, such antibodies, including antibody fragments can be produced by methods well known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and co-transfecting a recombinant host cell with the coding sequences for co-expression, using well known recombinant expression vectors. Recombinant host cells can be prokaryotic and eukaryotic cells, such as those described above.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol 151:2296 (1993); Chothia et al., J. Mol. Biol 196:901 (1987)). It is important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences.

In addition, human antibodies can be generated following methods known in the art. For example, transgenic animals (e.g., mice) can be made that are capable, upon immunization, of producing a full repertoire of human antibodies in the absence of endogenous immunoglobulin production. See, e.g., Jakobovits et al., Proc. Natl Acad. Sci. USA 90:2551 (1993); Jakobovits et al., Nature 362:255-258 (1993); Bruggermann et al., Year in Immuno. 7:33 (1993); and U.S. Pat. Nos. 5,591,669, 5,589,369 and 5,545,807.

Use of Neutralizing Antibodies

The influenza neutralizing antibodies of the present invention can be used for the prevention and/or treatment of influenza type A infections. For therapeutic applications, the antibodies or other molecules, the delivery of which is facilitated by using the antibodies or antibody-based transport sequences, are usually used in the form of pharmaceutical compositions. Techniques and formulations generally may be found in Remington's Pharmaceutical Sciences, 18th Edition, Mack Publishing Co. (Easton, Pa. 1990). See also, Wang and Hanson “Parenteral Formulations of Proteins and Peptides: Stability and Stabilizers,” Journal of Parenteral Science and Technology, Technical Report No. 10, Supp. 42-2S (1988).

Antibodies are typically formulated in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The antibodies also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, supra.

The neutralizing antibodies disclosed herein may also be formulated as immunoliposomes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257:286-288 (1982) via a disulfide interchange reaction. A chemotherapeutic agent is optionally contained within the liposome. See Gabizon et al. J. National Cancer Inst. 81(19)1484 (1989).

For the prevention or treatment of disease, the appropriate dosage of antibody will depend on the type of infection to be treated the severity and course of the disease, and whether the antibody is administered for preventive or therapeutic purposes. The antibody is suitably administered to the patient at one time or over a series of treatments. Depending on the type and severity of the disease, about 1 μg/kg to about 15 mg/kg of antibody is a typical initial candidate dosage for administration to the patient, whether, for example, by one or more separate administrations, or by continuous infusion.

Further details of the invention are illustrated by the following non-limiting Example.

### EXAMPLE

Antibody Libraries from Survivors of Prior Bird Flu Outbreaks and Preparation of Neutralizing Antibodies

Materials and Methods

Bone Marrow Protocol and Sera Preparation

Blood was obtained by standard venopuncture, allowed to clot and processed to recover serum. The serum was stored at −20° C. for 3-4 days until they were shipped on dry ice. Donors were anaesthetized with an injection of a local anesthetic and 5 ml of bone marrow was removed from the pelvic bone of each H5N1 survivor. Next the 5 ml of bone marrow was placed into a sterile 50-ml tube containing 45 ml RNAlater (Ambion). The mixture was gently inverted approximately 8-20 times, until there were no visible clumps and the marrow and RNAlater were mixed well. Next the specimen was refrigerated the between 2-10° C. overnight. Following the overnight refrigeration, the specimens were stored at −20° C. for 3-4 days until they were shipped on dry ice. Upon receipt the RNAlater/marrow and sera containing tubes were stored at −80° C. until processed.

Serology: HA ELISA

ELISA plates (Thermo, Immulon 4HBX 96W) were coated with 100 μl of 100 ng/mL H5 hemagglutinin (Protein Sciences, A/Vietnam/1203/2004) in 1× ELISA Plate Coating Solution (BioFX) by overnight incubation at room temperature. The next day plates were washed three times with 300 μl PBS/0.05% Tween-20 (PBST). Following the wash, 300 μl of a blocking solution (4% Non-Fat dry Milk in PBS/0.05% Tween-20) was added and incubated for 1 hour at RT. Following the blocking step, the plates were washed three times with 300 μl PBS/0.05% Tween-20. Next, 100 μl serum samples diluted 1:20,000 in PBS/0.05% Tween were incubated for 1-2 hours at RT and then washed three times with 300 μl PBS/0.05% Tween-20. 100 μl of an anti-human Fc-HRP conjugate diluted 1:5,000 in PBS/0.05% Tween was incubated for 1-2 hours at RT and then washed three times with 300 μl PBS/0.05% Tween-20. Following this final wash, 100 μl of chromogenic substrate solution was added (TMB1 Substrate, BioFx) and after sufficient amount of time terminated by the addition of 100 μl of STOP Solution (BioFx). Absorbances at 450 nm were read on a plate reader (Molecular Devices Thermomax microplate reader with Softmax Pro software), data recorded, and subsequently plotted using Excel (Microsoft).

Bone Marrow: RNA Extraction and mRNA Purification

Bone marrow (˜2.5 ml in 20 ml RNA later), previously stored at −80° C., was recovered by centrifugation to remove RNA later and then resuspended in 11.25 ml TRI BD reagent (Sigma) containing 300 μl Acetic Acid. The pellet was then vortexed vigorously. Next 1.5 ml BCP (1-bromo-3-chloropropane, Sigma) was added, mixed by vortexing, incubated at RT for 5 min, and then centrifuged at 12000×g for 15 min at 4° C. The aqueous phase was carefully removed to not disturb the interface. Total RNA from the aqueous phase was next precipitated by addition of 25 ml isopropanol, incubation at RT for 10 minutes, and centrifugation at 12000×g for 10 min at 4° C. Following the addition of isopropanol, two phases were formed due to residual RNAlater, resulting in the precipitated RNA settling at the interface. To eliminate the residual RNAlater and allow maximal recovery of RNA, 5 ml aliquots of 50% isopropanol in H₂O were added and mixed until no phase separation was noticeable, at which point the RNA was pelleted by centrifugation at 12000×g for 10 min at 4° C. The RNA pellet was washed with 75% EtOH, transferred to an RNAse-free 1.6 ml microcentrifuge tube, and again recovered by centrifugation. Finally the RNA pellet was resuspended in 100 μl 1 mM Na-phosphate, pH 8.2 and the A₂₆₀ and A₂₈₀ were read to assess RNA purity.

Prior to reverse transcription mRNA was purified from total RNA according to Qiagen Oligotex mRNA purification kit. Briefly, 50-200 μg bone marrow RNA was brought to 250 μl with RNase-free water and mixed with 250 μl of OBB buffer and Oligotex suspension followed by incubation for 3 min at 70° C. Hybridization between the oligo dT₃₀ of the Oligotex particle and the mRNA poly-A-tail was carried out at room temperature for 10 min. The hybridized suspensions were then transferred to a spin column and centrifuged for 1 min. The spin column was washed twice with 400 μl Buffer OW2. Purified mRNA was then eluted twice by centrifugation with 20 μl hot (70° C.) Buffer OEB. Typical yields were 500 ng to 1.5 μg total RNA.

Reverse Transcription Using N9 and Oligo dT on Bone Marrow mRNA

Reverse transcription (RT) reactions were accomplished by mixing together 75-100 ng mRNA with 2 μl 10× Accuscript RT Buffer (Stratagene), 0.8 μl 100 mM dNTPs, and either N9 (300 ng) or oligo dT primer (100 ng) and then brought to a final volume of 17 μl with water. The mixtures were heated at 65° C. for 5 min, and then allowed to cool to room temperature. Next 2 μl DTT, 0.5 μl RNase Block (Stratagene), 0.5 μl AccuScript RT (Stratagene) were added to each reaction. Next, the N9 primed reactions were incubated for 10 minutes at room temperature and the oligo-dT primed reactions were incubated on ice for 10 minutes. Finally, both reactions were incubated at 42° C. for 60 minutes followed by 70° C. for 15 minutes to kill the enzyme.

PCR From Bone Marrow-Derived cDNA

Antibody heavy and light chain repertoires were amplified from bone marrow cDNA essentially using previously described methods and degenerate primers (O'Brien, P. M., Aitken R. Standard protocols for the construction of scFv Libraries. Antibody Phage Display—Methods and Protocols, vol 178, 59-71, 2001, Humana Press) based upon human germline V and J regions.

Briefly, PCR reactions using Oligo dT primed cDNA (from 75 ng mRNA) for lambda light chains and N9 primed cDNA (from 75 ng mRNA for kappa light chains, from 100 ng mRNA for heavy chains) were mixed together with 5 μl 10× amplification buffer (Invitrogen), 1.5 μl dNTPs (10 mM), 1 μl MgSO4 (50 mM), 2.5 μl V_{region} primers (10 uM) and 2.5 μl J_{region} primers (10 uM) −10 uM for V_{H}, 0.5 μl Platinum Pfx Polymerase (Invitrogen), and sterile dH₂O to final volume of 50 μl. PCR parameters were as follows: step 1—95° C. 5 minutes, step 2—95° C. 30 seconds, step 3—58° C. 30 seconds, step 4—68° C. 1 minute, step 5—cycle step 2—4 40 times, step 6—68° C. 5 minutes. Light chain PCR products were cleaned up using Qiagen PCR Cleanup kit. Heavy chains PCR products were gel purified from 1.5% agarose gel using Qiagen Gel Extraction Kit and then reamplified. Heavy chain reamplification was carried out as follows: Mixed 10 μl 10× amplification buffer (Invitrogen), 3 μl dNTPs (10 mM), 2 μl MgSO4 (50 mM), 5 μl each V_{H} primers (10 uM) and J_{H} primers (10 uM), 5 μl Heavy chain Primary PCR product, 1 μl Platinum Pfx, volume adjusted to 100 μl with water. Cycling parameters were as follows: step 1—95° C. 5 minutes, step 2—95° C. 30 seconds, step 3—58° C. 30 seconds, step 4—68° C. 1 minute, step 5—cycle step 2—4 20 times, step 6—68° C. 5 minutes. Re-amplified heavy chain PCR products were cleaned up from a 1.5% agarose-TAE gel using Qiagen Extraction Kit.

Antibody Phage Library Construction

Separate antibody libraries for each individual bird flu survivor were constructed using unique identifying 3-nucleotide barcodes inserted in the untranslated region following the terminal pIII stop codon.

Light Chain Cloning:

1 μg each of pooled kappa light chain and pooled lambda light chain per donor were digested with NotI and BamHI and gel purified from a 1.5% agarose-TAE gel using Qiagen Gel Extraction Kit. 5 μg of each vector was digested with NotI and BamHI and gel purified from a 1% agarose-TAE gel using Qiagen Gel Extraction Kit. Library ligations were performed with 200 ng of gel purified Kappa or Lambda inserts and 1 μg of gel purified vector in 60 μl for 1 hour at RT or overnight at 14° C. Ligations were desalted using Edge BioSystem Perfroma spin columns. The library was transformed in five electroporations in 80 μl TG-1 or XL-1 Blue aliquots, each recovered in 1 ml SOC, pooled and outgrown for one hour at 37° C. Total number of transformants was determined following this outgrowth by plating an aliquot from each of the transformations. The remaining electroporation was amplified by growing overnight at 37° C. in 200 ml 2YT+50 μg/ml Ampicillin+2% glucose. The subsequent light chain library was recovered by plasmid purification from these overnight cultures using a Qiagen High Speed Maxiprep Kit.

Heavy Chain Cloning:

1.5-2 μg each of the donor-specific heavy chains (V_{H}1, V_{H}2, 5, 6 pool, V_{H}3, and V_{H}4) were digested with a 40 Unit excess/μg DNA with SfiI and XhoI and gel purified from a 1.5% agarose-TAE gel using Qiagen Gel Extraction Kit. 15 μg of each light chain library vector was digested with 40 Unit/μg DNA with SfiI and XhoI and gel purified from a 1% agarose-TAE gel using Qiagen Gel Extraction Kit. Library ligations were set up by combining 1.2 μg SfiI/XhoI digested, gel purified heavy chain donor collections and 5 μg of each light chain library (kappa and lambda) overnight at 14° C. The library ligations were then desalted with Edge BioSystem Pefroma spin columns and then transformed through 20 electroporations per library in 80 μl TG-1 aliquots, each recovered in 1 ml SOC, pooled and outgrown for one hour at 37° C. Again following this outgrowth an aliquot of each was used to determine the total number of transformants with the remainder transferred to 1L 2YT+50 μg/ml Ampicillin+2% glucose and grown at 37 C with vigorous aeration to an OD₆₀₀ of ˜0.3. Next M13K07 helper phage was then added at a multiplicity of infection (MOI) of 5:1 and incubated for 1 hour at 37° C., with no agitation. Next the cells were harvested by centrifugation and resuspended in 1L 2YT+50 μg/ml Ampicillin, 70 μg/ml Kanamycin and grown overnight at 37° C. with vigorous aeration to allow for scFv phagemid production. The next morning the cells were collected by centrifugation and supernatant containing phagemid was collected. The phagemids were precipitated from the supernatant by the addition of 0.2 volumes 20% PEG/5 M NaCl solution and incubation for 1 hour on ice. The phagemid library stocks were then harvested by centrifugation and resuspended in 20 ml sterile PBS. Residual bacteria were removed by an additional centrifugation and the final phagemid libraries were stored at −20° C. in PBS+50% glycerol.

Phagemid Panning and Amplification

ELISA plates (Immulon 4HBX flat bottom, Nunc) were coated with 100 μl of 100 ng/mL H5 hemagglutinin protein(Protein Sciences, A/Vietnam/1203/2004) in ELISA Coating Solution (BioFX) by overnight incubation at room temperature. The next day plates were washed three times with 300 μl PBST. Following the wash, 300 μl of a blocking solution (4% Non-Fat dry Milk in PBS/0.05% Tween-20) was added and incubated for 30 mins on ice. Following the blocking step, the plates were washed three times with 300 μl PBST. Just prior to phage panning, the glycerol was removed from the frozen phagemid stocks using Millipore Amicon Ultra columns and then blocked in 4% nonfat dry milk for 15 minutes. Next, 100 μl aliquots of phagemid were distributed into 8 wells (total phage ˜1×10¹² CFU) and incubated for 2 hours at 4° C. followed by washing 6-8 times with 300 μl PBST. Phagemid were collected following a 10 min at room temperature in 100 μl/well Elution buffer (0.2M glycine-HCl, pH 2.2, 1 mg/ml BSA). The eluate was then neutralized by the addition of 56.25 μl 2M Tris base per ml eluate. Following neutralization, 5 ml TG1 cells (OD₆₀₀˜0.3) were infected with 0.5 ml neutralized phage at 37° C. for 30 minutes in 2-YT with no shaking. Following this step some cells were plated onto LB AMP Glucose plates to determine total phagemid recovery. The remaining inoculum was placed into 10 ml 2-YTAG (final concentration 2% glucose and 50 ug/ml ampicillin) and grown at 37° C. with vigorous aeration to OD₆₀₀˜0.3. Next the cultures were infected with M13K07 helper phage at an MOI of 5:1 and incubated at 37° C. for 30-60 minutes with no shaking. The cells were collected by centrifugation and resuspended in 25 ml 2-YTAK (Ampicillin 50 μg/ml, Kanamycin 70 μg/ml), transferred to a fresh culture flask, and grown ON at 37° C. with shaking. Subsequent rounds were similarly recovered and amplified.

scFv ELISA

Individual colonies of E. coli HB2151 transformed cells from biopanned phage were grown overnight at 37° C. in 1 ml of 2YT+100 μg/ml AMP. The following morning the cells were harvested by centrifugation and resuspended in 1.5 ml periplasmic lysis buffer (1 ml BBS (Teknova)+0.5 ml 10 mg/ml lysozyme+EDTA to 10 mM final concentration). The cells were again pelleted by centrifugation and the scFv containing periplasmic lysates were collected. The scFv lysates were combined 1:1 with dilution buffer (PBS/0.05% BSA) and 100 μl was added to wells that had been previously antigen coated with and blocked with dilution buffer. The samples were incubated for 2 hours at room temperature and then washed three times with PBS/0.05% Tween. Next 100 μl of 1:5000 diluted Biotin Anti-Histidine mouse (Serotec) in dilution buffer was added to each well and incubated for 1 hr at room temperature. Following this incubation the wells were washed three times with PBS/0.05% Tween and then to each well 100 μl of 1:2500 Streptavidin:HRP (Serotec) was added and incubated for 1 hr at room temperature and then washed three times with PBS/0.05% Tween. Following this final wash, 100 μl of chromogenic substrate solution was added (TMB1 Substrate, BioFx) and after sufficient amount of time terminated by the addition of 100 μl of STOP Solution (BioFx). Absorbances at 450 nm were read on a plate reader (Molecular Devices Thermomax microplate reader with Softmax Pro software), data recorded, and subsequently plotted using Excel (Microsoft).

Sequencing

To deduce the heavy and light chain sequences, individual clones were grown and plasmid DNA extracted (Qiagen). The plasmid DNA was subjected to standard DNA sequencing.

Hemagglutinin Inhibition (HAI) Assays

Hemagglutination Inhibition was performed essentially following the method of Rogers et al., Virology 131:394-408 (1983), in round bottom microtiter plates (Corning) using 4 HAU (hemagglutinating units) of virus or protein/well. For HAI determinations 25 μl samples of purified single chain variable fragments (scFv) were mixed with 25 μl of PBS containing 4 HAU of the test virus in each microtiter well. Following a preincubation of 15 minutes at room temperature, 25 μl of 0.75% human erythrocytes were added, and mixed. HAI antibody activity was determined by visual inspection following a 60 min incubation at room temperature.

Results

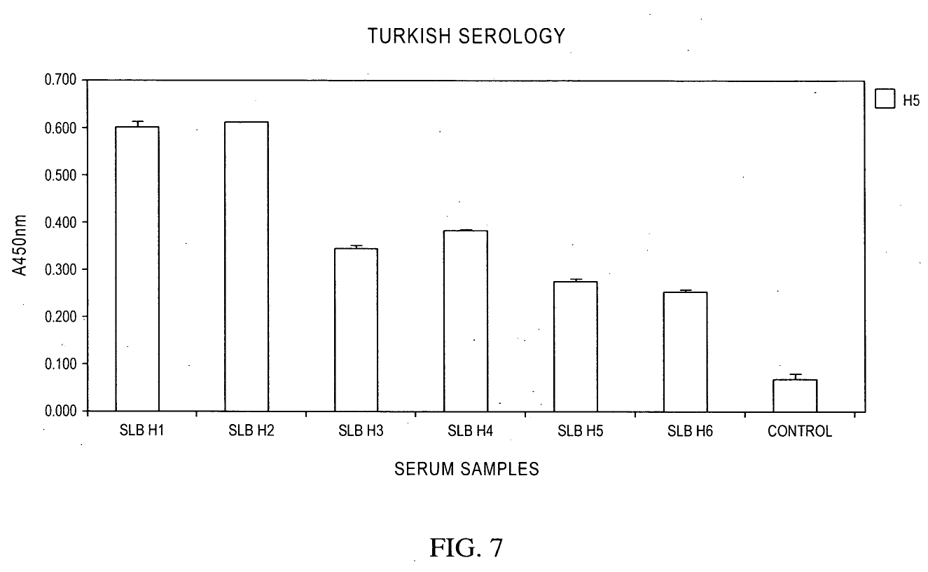

Bone marrow and blood samples were collected from six survivors of the H5 N1 bird flu outbreak that had taken place in Turkey in January 2006, approximately four months after the outbreak. For all six survivors the initial diagnosis of bird flu was made following by physical examination, clinical laboratory testing, and molecular diagnostic determination, sanctioned by the Turkish Ministry of Health. Four of these survivors were additionally confirmed by the World Health Organization (WHO). Serum samples were analyzed to confirm the presence of antibodies to H5 hemagglutinin (A/Vietnam/1203/2004) using the serology protocol described above. As shown in FIG. 7, the blood samples of all six patients (designated SLB H1-H6, respectively) demonstrated the presence of antibodies to the H5 antigen. Following this confirmation, RNA was extracted from the bone marrow samples of these individuals, and bone marrow mRNA was purified and reverse transcribed using the protocols described above. The antibody heavy and light chain repertoires were then amplified from the bone marrow cDNA as described above, and individual antibody heavy and light chain phage libraries were cloned separately for each survivor, using the above-described three-nucleotide bar coding to distinguish the individual libraries.

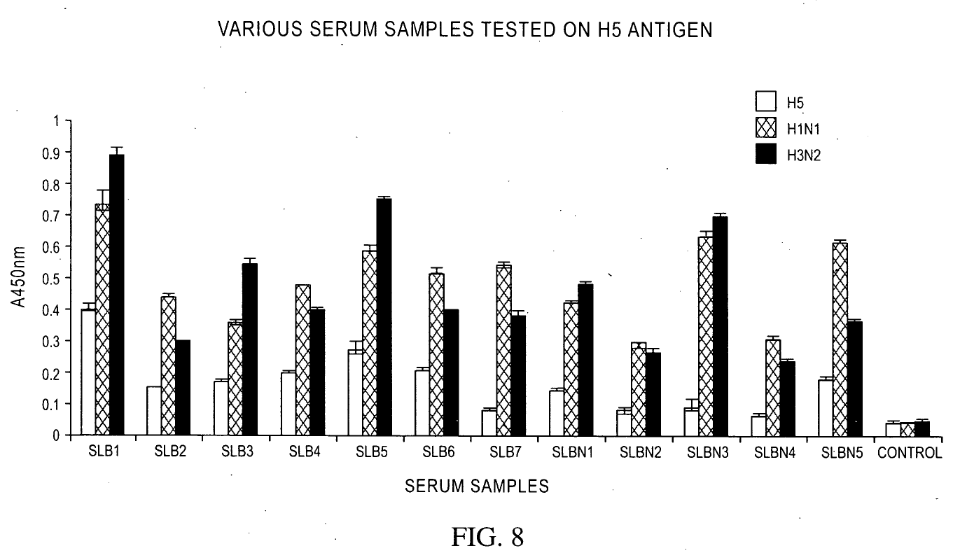

Bone marrow and blood samples were also collected from twelve local donors who were treated for flu symptoms in the year of 2006. Serology was performed as described above to confirm the presence of antibodies to H1, H3 and H5 hemagglutinin, respectively. As shown in FIG. 8, all serum samples tested positive for antibodies to H1 and/or H3 hemagglutinins, where the dominance of a certain subtype depended on the influenza A virus subtype to which the particular donor was exposed most throughout his or her lifetime. Interestingly, there were donors whose serum contained a significant level of antibodies of H5 hemagglutinin as well (donors SLB1 and SLB5 in FIG. 8). Following this confirmation, RNA was extracted from the bone marrow samples of the donors, and bone marrow mRNA was purified and reverse transcribed using the protocols described above. The antibody heavy and light chain repertoires were then amplified from the bone marrow cDNA as described above, and individual antibody heavy and light chain phage libraries were cloned separately for each donor, using the above-described three-nucleotide bar coding to distinguish the individual libraries.

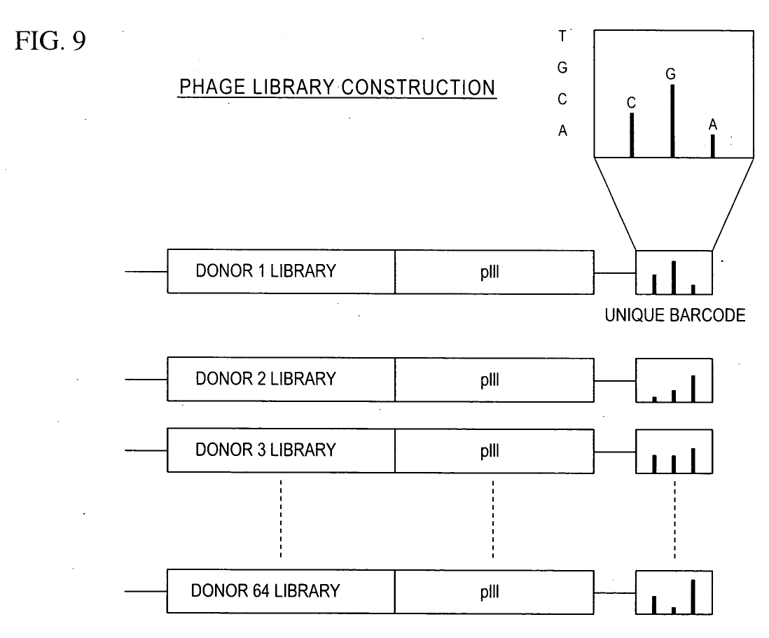

As illustrated in FIG. 9, using three of the available four nucleotides allows the creation of 64 unique barcodes.

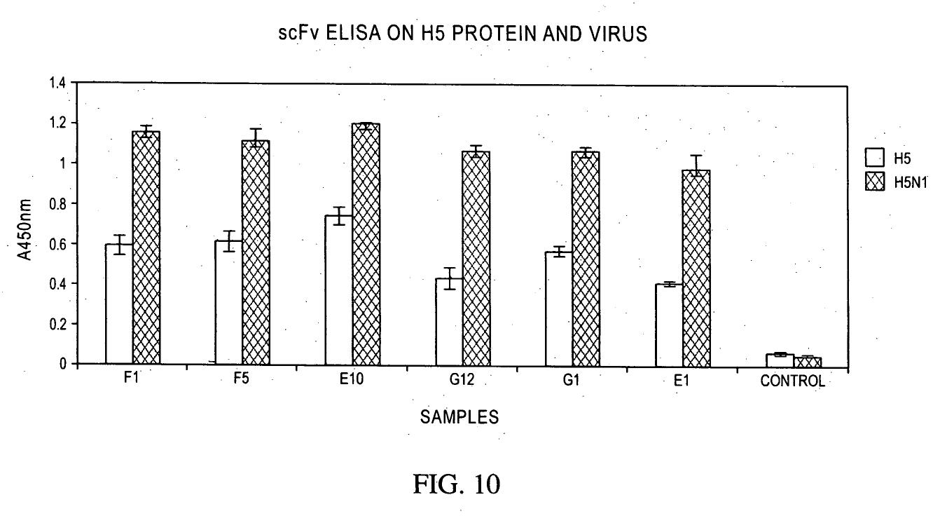

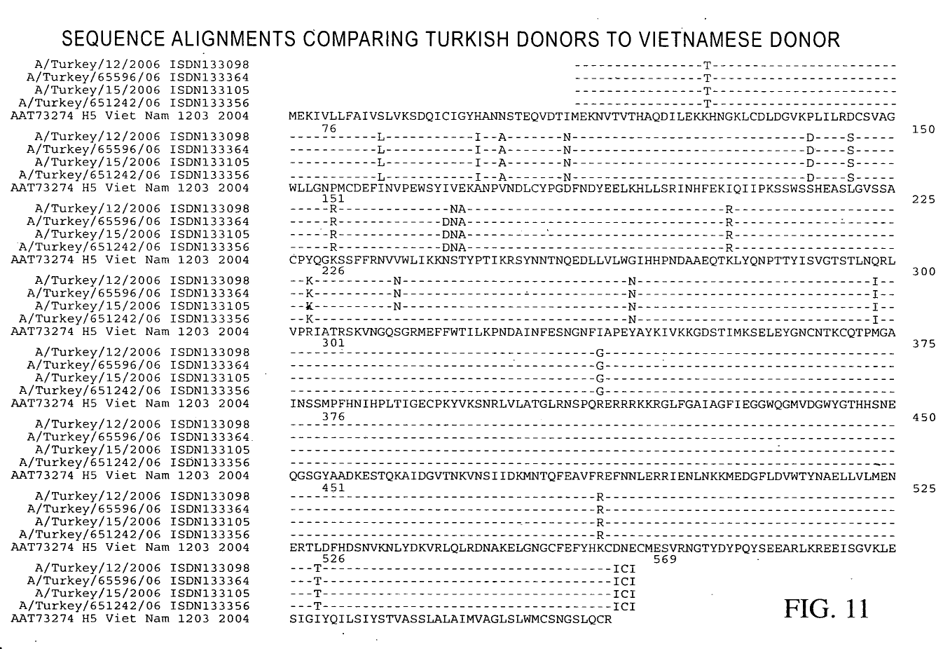

Out of 48 random clones obtained after three rounds of panning of pooled antibody libraries prepared from the bone marrow samples of Turkish bird flu survivors, 40 were tested by ELISA for binding to the H5 hemagglutinin protein (Protein Sciences, A/Vietnam/120312004), and to inactivated Vietnamese H5N1 virus (CBER, A/Vietnam/1203/2004). The clones were sequenced. Of the 40 clones, five were found to be different. As shown in FIG. 10, all five distinct clones (clones F5 and G1 have the same sequences) were binding both to the H5 protein and the Vietnamese H5N1 virus. FIG. 11 shows sequence alignments comparing the sequences of H5 hemagglutinin proteins from Turkish donors to the H5 hemagglutinin sequence of the Vietnamese isolate used in the above experiments. The results of these experiments show that, despite differences in the sequences, the antibodies tested bound both the Turkish and the Vietnamese H5 proteins and viruses, and thus showed cross-reactivity with more than one isolate of the H5N1 virus.

Four additional unique clones were identified from among 12 clones produced by the second round of panning.

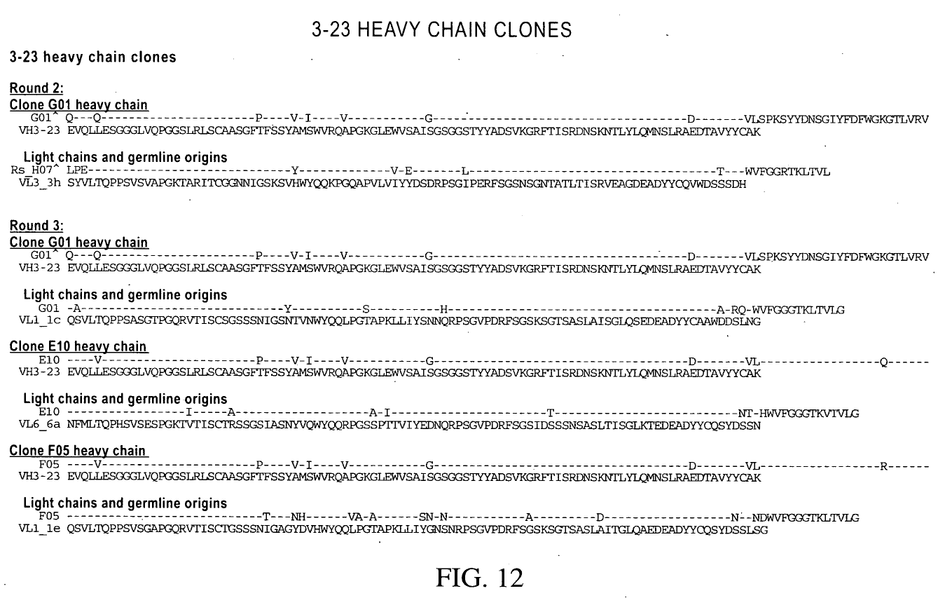

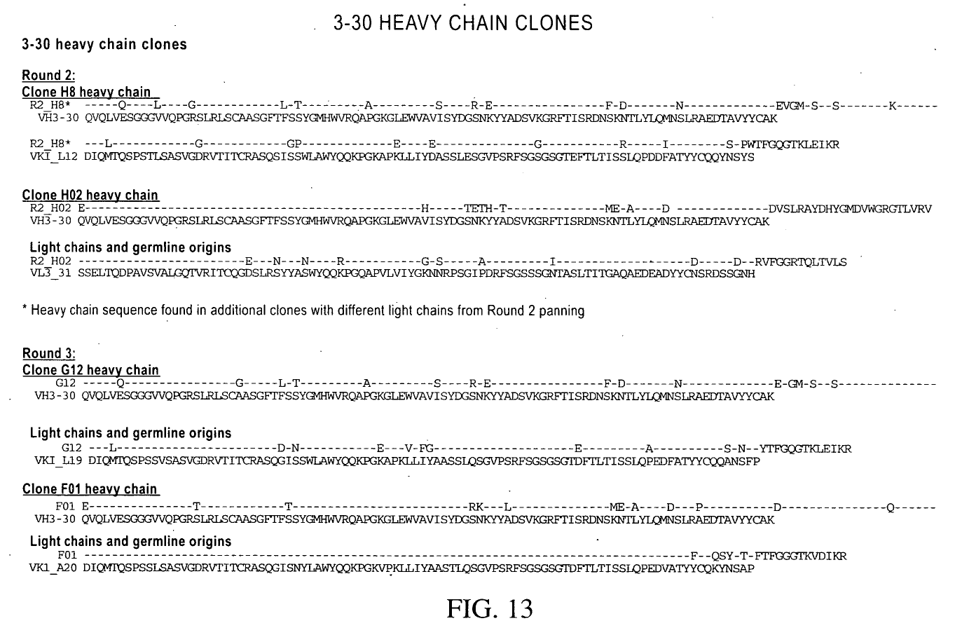

The heavy chain variable region sequences of the unique clones identified in the pooled antibody libraries of Turkish donors, along with the corresponding light chain and germline origin sequences, are shown in FIGS. 12 and 13. In particular, the sequences shown in FIG. 12 (3-23 heavy chain clones) originate from a pooled library of all heavy and light chains of all Turkish donors after three rounds of panning. The sequences shown in FIG. 13 (3-30 heavy chain clones) originate from a pooled library of all heavy and light chains of all Turkish donors after two rounds of panning.

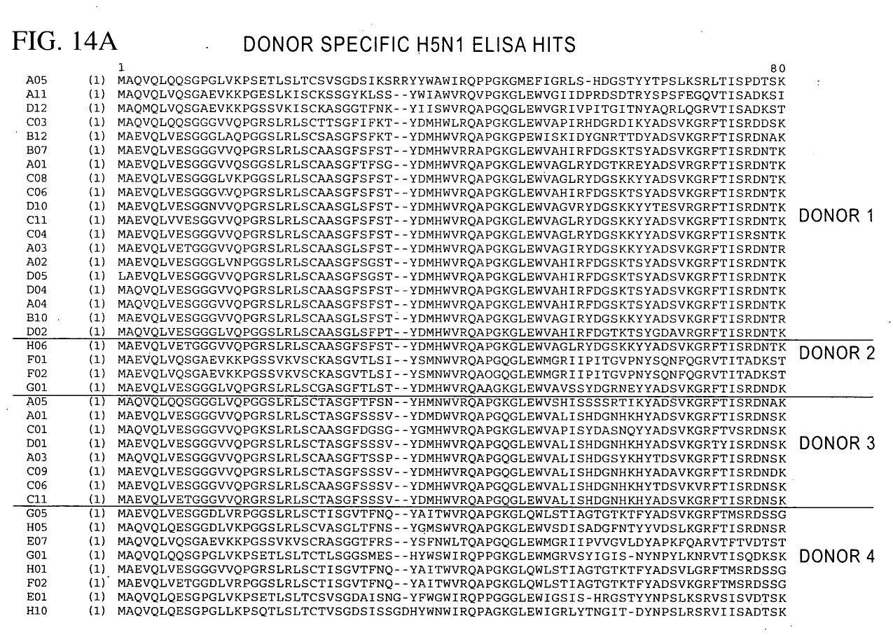

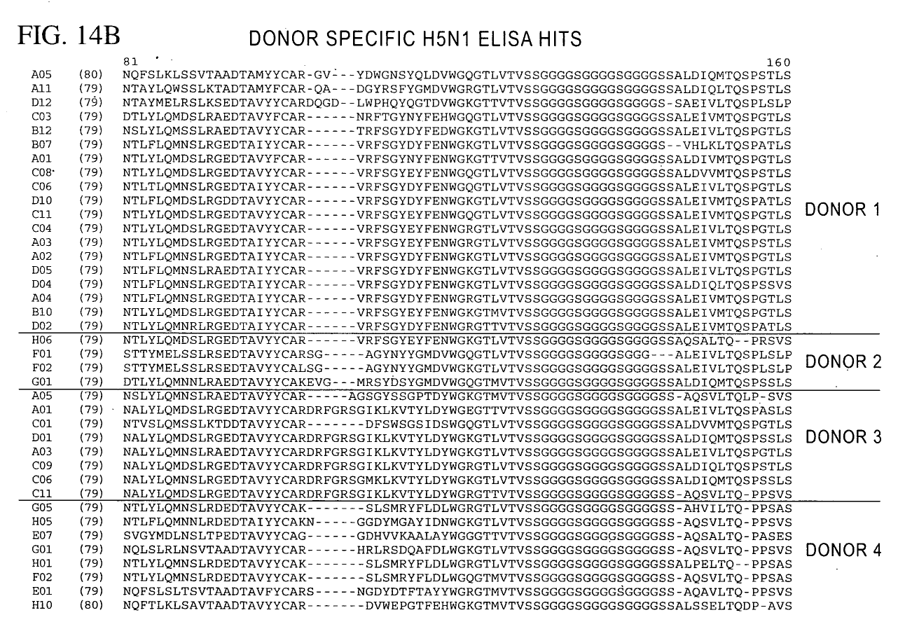

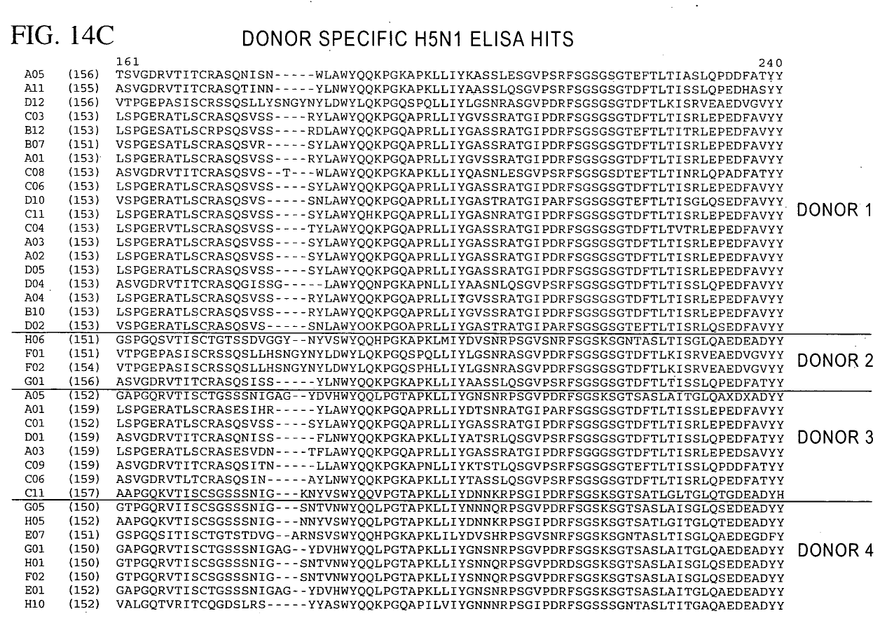

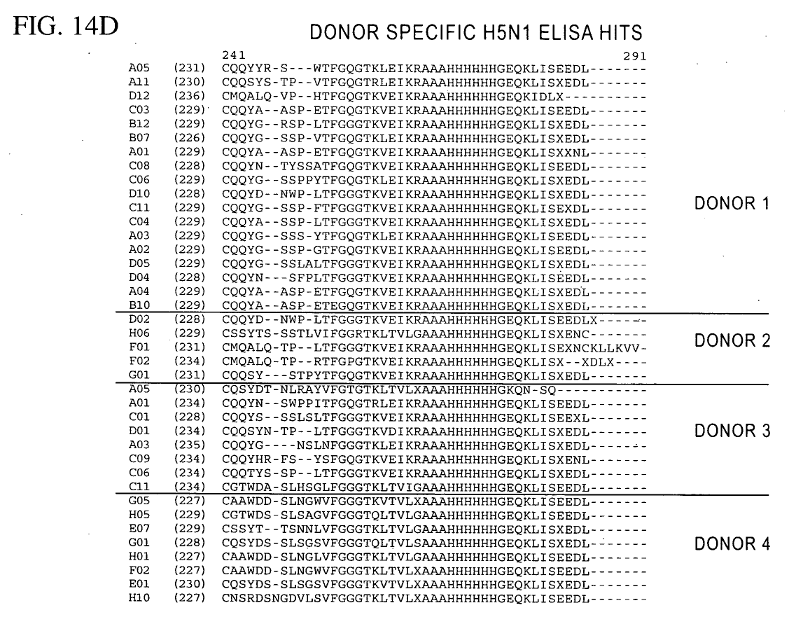

Additional unique H5N1 specific antibody heavy chain variable region sequences were identified from antibody libraries of individual Turkish donors, using the ELISA protocol described above, after four rounds of panning. The sequences of these H5N1 ELISA positive clones are shown in FIGS. 14A-D.

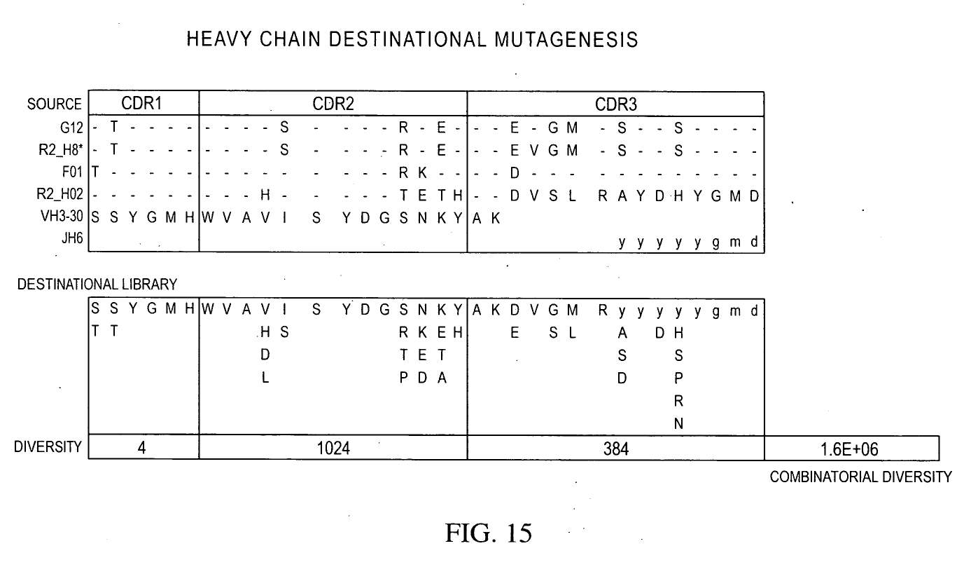

FIGS. 15 and 16 illustrate the use of destinational mutagenesis to create diverse antibody heavy and light chain libraries using the antibody heavy (FIG. 15) and light chain (FIG. 16) sequences identified by analysis of sera and bone marrow of Turkish bird flu survivors as described above.

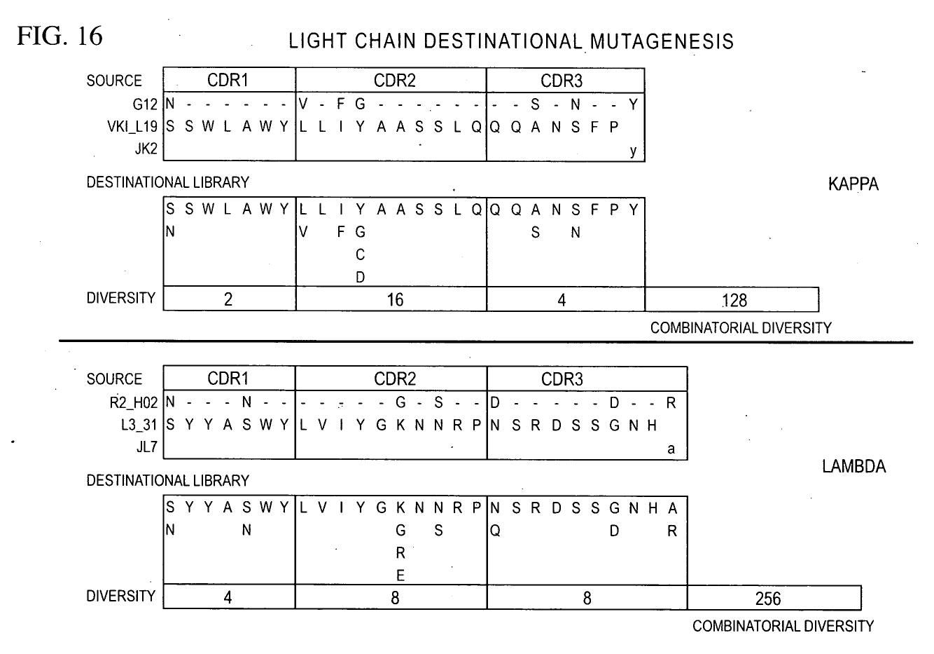

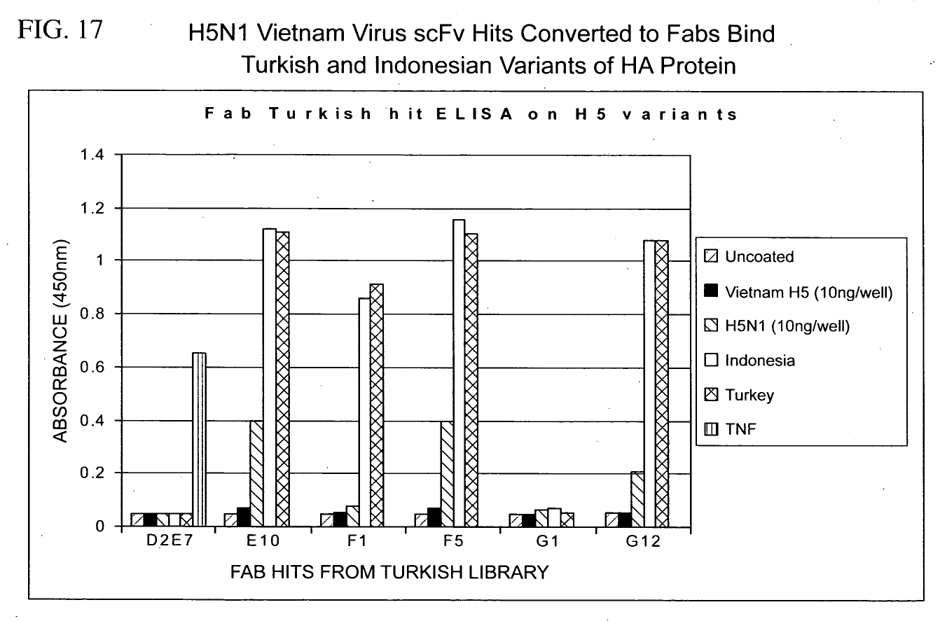

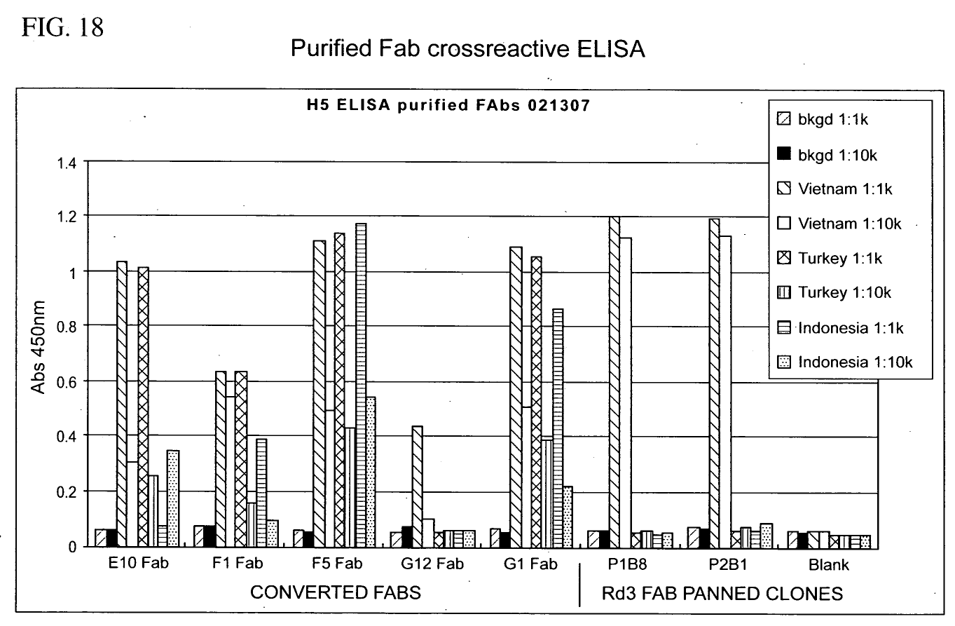

FIGS. 17 and 18 show ELISA results confirming cross-reactivity of certain Fab fragments obtained from an H5N1 Vietnam virus scFv antibody with Turkish and Indonesian variants of the HA protein.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

All references cited throughout the specification are hereby expressly incorporated by reference.

What is claimed is:

1. A neutralizing antibody neutralizing more than one isolate of an influenza A virus subtype and/or more than one subtype of the influenza A virus.

2. The neutralizing antibody of claim 1 neutralizing more than one isolate of influenza A virus H1 subtype.

3. The neutralizing antibody of claim 1 neutralizing more than one isolate of influenza A virus H3 subtype.

4. The neutralizing antibody of claim 1 neutralizing influenza A virus H1 and H3 subtypes.

5. The neutralizing antibody of claim 4 neutralizing more than one isolates of influenza A virus H1 and/or H3 subtypes.

6. The neutralizing antibody of claim 1 neutralizing substantially all isolates of an influenza A virus subtype.

7. The neutralizing antibody of claim 1 or claim 6 wherein said subtype is selected from the group consisting of H5, H7 and H9 subtypes.

8. The neutralizing antibody of claim 7 wherein said subtype is the H5 subtype.

9. The neutralizing antibody of claim 8 wherein said antibody neutralizes substantially all isolates of the influenza A virus H5 subtype.

10. The neutralizing antibody of claim 7 wherein said subtype is the H7 subtype.

11. The neutralizing antibody of claim 10 wherein said antibody neutralizes substantially all isolates of the influenza A virus H7 subtype.

12. The neutralizing antibody of claim 7 wherein said subtype is the H9 subtype.

13. The neutralizing antibody of claim 12 wherein said antibody neutralizes substantially all isolates of the influenza A virus H9 subtype.

14. The neutralizing antibody of claim 7 which further neutralizes at least one additional H subtype of influenza A virus.

15. The neutralizing antibody of claim 14 wherein said additional H subtype is selected from the group consisting of H1, H2 and H3 subtypes.

16. The neutralizing antibody of claim 15 neutralizing more than one isolate of said additional H subtype of influenza A virus.

17. The neutralizing antibody of claim 1 neutralizing the H5N1 subtype of influenza virus A.

18. The neutralizing antibody of claim 17 neutralizing more than one isolate of the H5N1 subtype of influenza virus A.

19. The neutralizing antibody of claim 18 wherein at least one of said isolates has the ability to infect humans.

20. The neutralizing antibody of claim 19 wherein at least one of said isolates has been obtained from a human subject.

21. The neutralizing antibody of claim 20 wherein said human subject is diseased.

22. The neutralizing antibody of claim 20 wherein said human subject recovered from infection with the H5N1 subtype of influenza virus A.

23. The neutralizing antibody of claim 19 wherein at least one of said isolates has been obtained from a non-human animal.

24. The neutralizing antibody of claim 23 wherein said non-human animal is a bird.

25. The neutralizing antibody of claim 24 wherein said non-human animal is a wild-fowl.

26. The neutralizing antibody of claim 24 wherein said non-human animal is a chicken.

27. The neutralizing antibody of claim 18 neutralizing substantially all isolates of the H5N 1 subtype of influenza virus A.

28. The neutralizing antibody of claim 17 neutralizing the H5N1 subtype and at least one additional subtype selected from the group consisting of H1N1, H2N2, and H3N2 subtypes.

29. The neutralizing antibody of claim 28 neutralizing more than isolates of the H5N1 subtype of influenza virus A.

30. The neutralizing antibody of claim 29 neutralizing substantially all isolates of the H5N1 subtype of influenza virus A.

31. The neutralizing antibody of claim 30 neutralizing more than one isolate of said additional subtype.

32. The neutralizing antibody of claim 31 neutralizing substantially all isolates of said additional subtype.

33. The neutralizing antibody of claim 1 wherein said antibody binds to an H5 protein.

34. The neutralizing antibody of claim 33 wherein said antibody binds to more than one variant of the H5 protein.

35. The neutralizing antibody of claim 34 wherein said antibody binds to all variants of the H5 protein.

36. The neutralizing antibody of claim 35 wherein said antibody binds to at least one additional H protein.

37. The neutralizing antibody of claim 36 wherein said additional H protein is selected from the group consisting of H1, H2, and H3 proteins.

38. The neutralizing antibody of claim 37 wherein said antibody binds to more than one variant of said additional H protein.

39. The neutralizing antibody of claim 38 wherein said antibody binds to substantially all variants of said additional H protein.

40. A composition comprising a neutralizing antibody according to any one of claims 1-39.

41. A method for identifying an antibody capable of neutralizing more than one isolate of an influenza A virus subtype or more than one subtype of an influenza A virus, comprising identifying, in an antibody library, antibodies that react with both a first and a second isolate of said influenza A virus subtype or with a first and a second subtype of said influenza A virus, and subjecting the antibodies identified to successive alternating rounds of selection, based on their ability to bind said first and second isolates, or said first and second subtypes, respectively.

42. The method of claim 41 comprising at least two rounds of selection.

43. The method of claim 41 wherein said first and second isolates are different isolates of the H5N1 subtype of said influenza A virus.

44. The method of claim 41 wherein said antibodies that react with both a first and a second influenza A virus subtype isolate have been identified by at least two rounds of separate enrichment of antibodies reacting with the first isolate and the second isolate, respectively, and recombining the antibodies identified.

45. The method of claim 41 wherein said antibody that can react with both said first and said second influenza A subtype isolate is subjected to mutagenesis prior to being subjected to said successive alternating rounds of selection, based on their ability to bind said first and second isolate, respectively.

46. The method of claim 41 wherein said antibody library is a phage display library.

47. The method of claim 46 wherein selection is performed by biopanning.

48. The method of claim 41 wherein said influenza A virus subtype is an H5N1 subtype.

49. The method of claim 48 wherein said first isolate in a 2006 Turkish isolate of the H5N1 virus.

50. The method of claim 48 wherein said first isolate is a 2003/2004 Vietnam isolate of the H5N1 virus.

51. The method of claim 48 wherein said second isolate is a 2003/2004 Vietnam isolate of the H5N1 virus.

52. The method of claim 50 wherein said second isolate is a 1997 Hong Kong isolate of the H5N1 virus.

53. The method of claim 48 wherein said first and said second isolates originate from different species.

54. The method of claim 53 wherein at least one of said species is human.

55. The method of claim 53 wherein at least one of said species is a bird.

56. The method of claim 41 wherein said antibodies capable of binding said first and said second isolates are additionally selected based on their ability to bind more than one influenza A subtype.

57. A collection of sequences shared by the neutralizing antibodies identified by the method of any one of claims 41 to 56.

58. A collection of sequences comprising one or more of the unique heavy and/or light chain sequences shown in FIGS. 11, 12, 13, and 14A-D or a consensus or variant sequence based on said sequences.

59. A neutralizing antibody identifiable by the method of any one of claims 41 to 56, or a fragment thereof.

60. The neutralizing antibody of claim 59 comprising a heavy and/or light chain sequence selected from the unique sequences shown in FIGS. 11, 12, 13, and 14A-D, or a consensus or variant sequence based on said sequences, or a fragment thereof.

61. The neutralizing antibody or antibody fragment of claim 59 or claim 60 capable of conferring passive immunity to an avian or mammalian subject against an influenza A virus infection.

62. The neutralizing antibody or antibody fragment of claim 61 wherein said mammalian subject is a human.

63. The neutralizing antibody or antibody fragment of claim 62 wherein said influenza A virus infection is caused by a virus selected from the group consisting one H5N1, H1N1, H2N2, and H3N2 subtypes.

64. A method for the prevention and/or treatment of an influenza A infection in a subject comprising administering to said subject an effective amount of a composition of claim 40.

65. A method for treating influenza A infection in a subject comprising administering to said subject an effective amount of a neutralizing antibody of claim 59.

66. The method of claim 64 or claim 65 wherein said subject is a human patient.

67. A method for preventing influenza A infection comprising administering to a subject at risk of developing influenza A infection an effective amount of a composition of claim 40.

68. A method for preventing influenza A infection comprising administering to a subject at risk of developing influenza A infection an effective amount of a neutralizing antibody of claim 59.

69. The method of claim 67 or claim 68 wherein said subject is a human patient.

70. A method for producing a diverse multifunctional antibody collection, comprising (a) aligning CDR sequences of at least two functionally different antibodies, (b) identifying amino acid residues conserved between the CDR sequences aligned, (c) performing mutagenesis of multiple non-conserved amino acid residues in at least one of the CDR sequences aligned, using degenerate oligonucleotide probes encoding at least the amino acid residues present in the functionally different antibodies at the non-conserved positions mutagenized to produce multiple variants of the aligned CDR sequences, and, if desired, repeating steps (b) and (c) with one or more of said variants until said antibody collection reaches a desired degree of diversity or size.

71. The method of claim 70 wherein the CDR sequences aligned have the same lengths.

72. The method of claim 70 wherein the mutagenized variants produced in step (c) retain all conserved residues present in at least two of the CDR sequences aligned.

73. The method of claim 70 wherein the mutagenized variants produced in step (c) retain all conserved residues present in all of the CDR sequences aligned.

74. The method of claim 70 wherein said functionally different antibodies bind to different epitopes on a target antigen.

75. The method of claim 70 wherein said functionally different antibodies bind to different target antigens.

76. The method of claim 75 wherein said different target antigens are variants of the same antigen.

77. The method of claim 70 wherein said functionally different antibodies have different binding affinities.

78. The method of claim 70 wherein said functionally different antibodies have different biological properties.

79. The method of claim 70 wherein said functionally different antibodies bind to an influenza A virus.

80. The method of claim 79 wherein at least two of said functionally different antibodies bind to different epitopes on the same influenza A virus.

81. The method of claim 79 wherein said functionally different antibodies bind to different influenza A virus subtypes.

82. The method of claim 79 wherein at least two of said functionally different antibodies bind to different isolates of the same influenza A virus subtype.

83. The method of claim 79 wherein at least two of said functionally different antibodies bind to different isolates of the same influenza A virus subtype and different influenza A virus subtypes.

84. The method of any one of claims 70 to 83 wherein at least two of said functionally different antibodies have different binding affinities.

85. The method of any one of claims 70 to 83 wherein at least two of said functionally different antibodies differ in their ability to neutralize the influenza A virus to which they bind.

86. An antibody collection comprising a plurality of neutralizing antibodies which differ from each other in at least one property.

87. The antibody collection of claim 86 which comprises at least about 100 neutralizing antibodies.

88. The antibody collection of claim 87 prepared by the method of any one of claims 70 to 83.

89. A method for uniquely identifying nucleic acids in a collection comprising labeling said nucleic acids with a unique barcode linked to or incorporated in the sequences of the nucleic acid present in said collection.

90. The method of claim 89 wherein said barcode is a noncoding nucleotide sequence of one to about 24 nucleotides in length.

91. The method of claim 90 wherein said noncoding nucleotide sequence is linked to the 3′ noncoding region of the nucleic acid sequences labeled.

92. The method of claim 89 wherein said barcode is the coding sequence of one or more silent mutations incorporated into the nucleic acid sequences labeled.

93. The method of claim 89 wherein said barcode is a peptide or polypeptide sequence.
